(19) United States
(12) Patent Application Publication
(10) Pub. No.: US 2003/0064000 A1
(43) Pub. Date: Apr. 3, 2003

(54) Methods of sterilizing biological mixtures using stabilizer mixtures

(76) Inventors: Wilson Burgess, Clifton, VA (US); William N. Drohan, Springfield, VA (US); Martin J. MaCphee, Montgomery Village, MD (US); David M. Mann, Gaithersburg, MD (US)

Correspondence Address: FLESHNER & KIM LLP, P.O. Box 221200, Chantilly, VA, 20153-1200, US

(21) Appl. No.: 09/960,700

(22) Filed: Sep. 24, 2001

(51) Int. Cl. A61L 2/08; A01N 1/02

(52) U.S. Cl. 422/22; 422/23; 422/24; 435/2

(57) ABSTRACT

Methods are disclosed for sterilizing biological materials to reduce the level of one or more biological contaminants or pathogens therein, such as viruses, bacteria (including inter- and intracellular bacteria, such as mycoplasmas, ureaplasmas, nanobacteria, chlamydia, rickettsias), yeasts, molds, fungi, single or multicellular parasites, and/or prions or similar agents responsible, alone or in combination, for TSEs. These methods involve the use of stabilizer mixtures in methods of sterilizing biological materials with irradiation.

### BACKGROUND OF THE INVENTION

1. Field of the Invention

The present invention relates to methods for sterilizing biological materials to reduce the level of one or more biological contaminants or pathogens therein, such as viruses, bacteria (including inter- and intracellular bacteria, such as mycoplasmas, ureaplasmas, nanobacteria, chlamydia, rickettsias), yeasts, molds, fungi, single or multicellular parasites, and/or prions or similar agents responsible, alone or in combination, for TSEs. The present invention particularly relates to the use of stabilizer mixtures in methods of sterilizing biological materials with irradiation.

2. Background of the Related Art

Many biological materials that are prepared for human, veterinary, diagnostic and/or experimental use may contain unwanted and potentially dangerous biological contaminants or pathogens, such as viruses, bacteria (including inter- and intracellular bacteria, such as mycoplasmas, ureaplasmas, nanobacteria, chlamydia, rickettsias), yeasts, molds, fungi, single or multicellular parasites, and/or prions or similar agents responsible, alone or in combination, for TSEs. Consequently, it is of utmost importance that any biological contaminant in the biological material be inactivated before the product is used. This is especially critical when the material is to be administered directly to a patient, for example in blood transfusions, blood factor replacement therapy, organ transplants and other forms of human therapy corrected or treated by intravenous, intramuscular or other forms of injection or introduction. This is also critical for the various biological materials that are prepared in media or via culture of cells or recombinant cells which contain various types of plasma and/or plasma derivatives or other biologic materials and which may contain prions, bacteria, viruses and other biological contaminants or pathogens.

Most procedures for producing biological materials have involved methods that screen or test the biological materials for one or more particular biological contaminants or pathogens rather than removal or inactivation of the contaminant(s) and/or pathogen(s) from the material. Materials that test positive for a biological contaminant or pathogen are merely not used. Examples of screening procedures include the testing for a particular virus in human blood from blood donors. Such procedures, however, are not always reliable and are not able to detect the presence of certain viruses, particularly in very low numbers. This reduces the value or certainty of the test in view of the consequences associated with a false negative result. False negative results can be life threatening in certain cases, for example in the case of Acquired Immune Deficiency Syndrome (AIDS). Furthermore, in some instances it can take weeks, if not months, to determine whether or not the material is contaminated. Moreover, to date, there is no reliable test or assay for identifying prions within a biological material that is suitable for screening out potential donors or infected material. This serves to heighten the need for an effective means of destroying prions within a biological material, while still retaining the desired activity of that material. Therefore, it would be desirable to apply techniques that would kill or inactivate biological contaminants and pathogens during and/or after manufacturing the biological material.

The importance of these techniques is apparent regardless of the source of the biological material. All living cells and multi-cellular organisms can be infected with viruses and other pathogens. Thus the products of unicellular natural or recombinant organisms or tissues carry a risk of pathogen contamination. In addition to the risk that the producing cells or cell cultures may be infected, the processing of these and other biological materials creates opportunities for environmental contamination. The risks of infection are more apparent for multicellular natural and recombinant organisms, such as transgenic animals. Interestingly, even products from species as different from humans as transgenic plants carry risks, both due to processing contamination as described above, and from environmental contamination in the growing facilities, which may be contaminated by pathogens from the environment or infected organisms that co-inhabit the facility along with the desired plants. For example, a crop of transgenic corn grown out of doors, could be expected to be exposed to rodents such as mice during the growing season. Mice can harbour serious human pathogens such as the frequently fatal Hanta virus. Since these animals would be undetectable in the growing crop, viruses shed by the animals could be carried into the transgenic material at harvest. Indeed, such rodents are notoriously difficult to control, and may gain access to a crop during sowing, growth, harvest or storage. Likewise, contamination from overflying or perching birds has the potential to transmit such serious pathogens as the causative agent for psittacosis. Thus any biological material, regardless of its source, may harbour serious pathogens that must be removed or inactivated prior to the administration of the material to a recipient.

In conducting experiments to determine the ability of technologies to inactivate viruses, the actual viruses of concern are seldom utilized. This is a result of safety concerns for the workers conducting the tests, and the difficulty and expense associated with the containment facilities and waste disposal. In their place, model viruses of the same family and class are used.

In general, it is acknowledged that the most difficult viruses to inactivate are those with an outer shell made up of proteins, and that among these, the most difficult to inactivate are those of the smallest size. This has been shown to be true for gamma irradiation and most other forms of radiation as these viruses' diminutive size is associated with a small genome. The magnitude of direct effects of radiation upon a molecule are directly proportional to the size of the molecule, that is the larger the target molecule, the greater the effect. As a corollary, it has been shown for gamma-irradiation that the smaller the viral genome, the higher the radiation dose required to inactive it.

Among the viruses of concern for both human and animal-derived biological materials, the smallest, and thus most difficult to inactivate, belong to the family of Parvoviruses and the slightly larger protein-coated Hepatitis virus. In humans, the Parvovirus B19, and Hepatitis A are the agents of concern. In porcine-derived materials, the smallest corresponding virus is Porcine Parvovirus. Since this virus is harmless to humans, it is frequently chosen as a model virus for the human B19 Parvovirus. The demonstration of inactivation of this model parvovirus is considered adequate proof that the method employed will kill human B19 virus and Hepatitis A, and by extension, that it will also kill the larger and less hardy viruses such as HIV, CMV, Hepatitis B and C and others.

More recent efforts have focussed on methods to remove or inactivate contaminants in the products. Such methods include heat treating, filtration and the addition of chemical inactivants or sensitizers to the product.

Heat treatment requires that the product be heated to approximately 60EC for about 70 hours which can be damaging to sensitive products. In some instances, heat inactivation can actually destroy 50% or more of the biological activity of the product.

Filtration involves filtering the product in order to physically remove contaminants. Unfortunately, this method may also remove products that have a high molecular weight. Further, in certain cases, small viruses may not be removed by the filter.

The procedure of chemical sensitization involves the addition of noxious agents which bind to the DNA/RNA of the virus and which are activated either by UV or other radiation. This radiation produces reactive intermediates and/or free radicals which bind to the DNA/RNA of the virus, break the chemical bonds in the backbone of the DNA/RNA, and/or cross-link or complex it in such a way that the virus can no longer replicate. This procedure requires that unbound sensitizer is washed from products since the sensitizers are toxic, if not mutagenic or carcinogenic, and cannot be administered to a patient.

Irradiating a product with gamma radiation is another method of sterilizing a product. Gamma radiation is effective in destroying viruses and bacteria when given in high total doses (Keathly et al., “Is There Life After Irradiation? Part 2,” BioPharm July-August, 1993, and Leitman, Use of Blood Cell Irradiation in the Prevention of Post Transfusion Graft-vs-Host Disease,” Transfusion Science 10:219-239 (1989)). The published literature in this area, however, teaches that gamma radiation can be damaging to radiation sensitive products, such as blood, blood products, protein and protein-containing products. In particular, it has been shown that high radiation doses are injurious to red cells, platelets and granulocytes (Leitman). U.S. Pat. No. 4,620,908 discloses that protein products must be frozen prior to irradiation in order to maintain the viability of the protein product. This patent concludes that “[i]f the gamma irradiation were applied while the protein material was at, for example, ambient temperature, the material would be also completely destroyed, that is the activity of the material would be rendered so low as to be virtually ineffective”. Unfortunately, many sensitive biological materials, such as monoclonal antibodies (Mab), may lose viability and activity if subjected to freezing for irradiation purposes and then thawing prior to administration to a patient.

In view of the difficulties discussed above, there remains a need for methods of sterilizing compositions containing one or more biological materials that are effective for reducing the level of active biological contaminants or pathogens without an adverse effect on the material(s).

The above references are incorporated by reference herein where appropriate for appropriate teachings of additional or alternative details, features and/or technical background.

### SUMMARY OF THE INVENTION

An object of the invention is to solve at least the related art problems and disadvantages, and to provide at least the advantages described hereinafter.

Accordingly, it is an object of the present invention to provide methods of sterilizing biological compositions by reducing the level of active biological contaminants or pathogens without adversely affecting the composition. Other objects, features and advantages of the present invention will be set forth in the detailed description of preferred embodiments that follows, and in part will be apparent from the description or may be learned by practice of the invention. These objects and advantages of the invention will be realized and attained by the compositions and methods particularly pointed out in the written description and claims hereof.

In accordance with these and other objects, a first embodiment of the present invention is directed to a method for sterilizing a biological material that is sensitive to radiation comprising: (i) adding to a biological material at least one stabilizer mixture in an amount effective to protect the biological material from radiation; and (ii) irradiating the biological material with radiation at an effective rate for a time effective to sterilize the biological material.

Another embodiment of the present invention is directed to a method for sterilizing a biological material that is sensitive to radiation comprising: (i) reducing the residual solvent content of a biological material; (ii) adding to the biological material at least one stabilizer mixture; and (iii) irradiating the biological material with radiation at an effective rate for a time effective to sterilize the biological material, wherein the level of residual solvent content and the amount of stabilizer mixture are together effective to protect the biological material from radiation. According to this embodiment, steps (i) and (ii) may be reversed.

Another embodiment of the present invention is directed to a method for sterilizing a biological material that is sensitive to radiation comprising: (i) reducing the temperature of a biological material; (ii) adding to the biological material at least one stabilizer mixture; and (iii) irradiating the biological material with radiation at an effective rate for a time effective to sterilize the biological material, wherein the temperature and the amount of stabilizer mixture are together effective to protect the biological material from radiation. According to this embodiment, steps (i) and (ii) may be reversed.

Another embodiment of the present invention is directed to a method for sterilizing a biological material that is sensitive to radiation comprising: (i) reducing the residual solvent content of a biological material; (ii) adding to the biological material at least one stabilizer mixture; (iii) reducing the temperature of the biological material; and (iv) irradiating the biological material with radiation at an effective rate for a time effective to sterilize the biological material, wherein the temperature and the amount of stabilizer mixture are together effective to protect the biological material from radiation. According to this embodiment, steps (i), (ii) and (iii) may be preformed in any order.

The present invention also provides a biological composition comprising at least one biological material and at least one stabilizer mixture in an amount effective to protect the biological material for its intended use following sterilization with radiation.

The present invention also provides a biological composition comprising at least one biological material and at least one stabilizer mixture, in which the residual solvent content has been reduced to a level effective to protect the biological material for its intended use following sterilization with radiation.

The present invention also provides a biological composition comprising at least one biological material and at least one stabilizer mixture in which the residual solvent content has been reduced and wherein the amount of stabilizer mixture and level of residual solvent content are together effective to protect the biological material for its intended use following sterilization with radiation.

Additional advantages, objects, and features of the invention will be set forth in part in the description which follows and in part will become apparent to those having ordinary skill in the art upon examination of the following or may be learned from practice of the invention. The objects and advantages of the invention may be realized and attained as particularly pointed out in the appended claims.

### BRIEF DESCRIPTION OF THE DRAWINGS

The invention will be described in detail with reference to the following drawings in which like reference numerals refer to like elements wherein:

FIGS. 1A and 1B show the protective effect of ascorbate (200 mM), alone or in combination with Gly-Gly (200 mM), on a liquid polyclonal antibody preparation.

FIGS. 2A and 2B show the protective effect of the combination of ascorbate (200 mM) and Gly-Gly (200 mM) on two different frozen enzyme preparations (a galactosidase and a sulfatase).

FIG. 3 shows the protective effect of the combination of ascorbate (200 mM) and Gly-Gly (200 mM) on a frozen galactosidase preparation.

FIG. 4 shows the protective effect of 1.5 mM uric acid in the presence of varying amounts of ascorbate on gamma irradiated immobilized anti-insulin monoclonal antibodies.

FIG. 5 shows the protective effects of 2.25 mM uric acid in the presence of varying amounts of ascorbate on gamma irradiated immobilized anti-insulin monoclonal antibodies.

FIG. 6 shows the protective effects of the combination of ascorbate (200 mM) and Gly-Gly (200 mM) on lyophilized galactosidase preparations.

### DETAILED DESCRIPTION OF PREFERRED EMBODIMENTS

A. Definitions

Unless defined otherwise, all technical and scientific terms used herein are intended to have the same meaning as is commonly understood by one of ordinary skill in the relevant art.

As used herein, the singular forms “a,” “an,” and “the” include the plural reference unless the context clearly dictates otherwise.

As used herein, the term “biological material” is intended to mean any substance derived or obtained from a living organism. Illustrative examples of biological materials include, but are not limited to, the following: cells; tissues; blood or blood components; proteins, including recombinant and transgenic proteins, and proteinaceous materials; enzymes, including digestive enzymes, such as trypsin, chymotrypsin, alpha-glucosidase and iduronodate-2-sulfatase; immunoglobulins, including mono and polyimmunoglobulins; botanicals; food; and the like. Preferred examples of biological materials include, but are not limited to, the following: ligaments; tendons; nerves; bone, including demineralized bone matrix, grafts, joints, femurs, femoral heads, etc.; teeth; skin grafts; bone marrow, including bone marrow cell suspensions, whole or processed; heart valves; cartilage; corneas; arteries and veins; organs, including organs for transplantation, such as hearts, livers, lungs, kidneys, intestines, pancreas, limbs and digits; lipids; carbohydrates; collagen, including native, afibrillar, atelomeric, soluble and insoluble, recombinant and transgenic, both native sequence and modified; enzymes; chitin and its derivatives, including NO-carboxy chitosan (NOCC); stem cells, islet of Langerhans cells and other cells for transplantation, including genetically altered cells; red blood cells; white blood cells, including monocytes; and platelets.

As used herein, the term “sterilize” is intended to mean a reduction in the level of at least one active or potentially active biological contaminant or pathogen found in the biological material being treated according to the present invention.

As used herein, the term “biological contaminant or pathogen” is intended to mean a contaminant or pathogen that, upon direct or indirect contact with a biological material, may have a deleterious effect on a biological material or upon a recipient thereof. Such biological contaminants or pathogens include the various viruses, bacteria (including inter- and intracellular bacteria, such as mycoplasmas, ureaplasmas, nanobacteria, chlamydia, rickettsias), yeasts, molds, fungi, single or multicellular parasites, and/or prions or similar agents responsible, alone or in combination, for TSEs known to those of skill in the art to generally be found in or infect biological materials. Examples of biological contaminants or pathogens include, but are not limited to, the following: viruses, such as human immunodeficiency viruses and other retroviruses, herpes viruses, filoviruses, circoviruses, paramyxoviruses, cytomegaloviruses, hepatitis viruses (including hepatitis A, B and C and variants thereof), pox viruses, toga viruses, Epstein-Barr viruses and parvoviruses; bacteria (including mycoplasmas, ureaplasmas, nanobacteria, chlamydia, rickettsias), such as Escherichia, Bacillus, Campylobacter, Streptococcus and Staphylococcus; parasites, such as Trypanosoma and malarial parasites, including Plasmodium species; yeasts; molds; and prions, or similar agents, responsible alone or in combination for TSE (transmissible spongiform encephalopathies), such as scrapie, kuru, BSE (bovine spongiform encephalopathy), CJD (Creutzfeldt-Jakob disease), Gerstmann-Straeussler-Scheinkler syndrome, and fatal familial insomnia. As used herein, the term “active biological contaminant or pathogen” is intended to mean a biological contaminant or pathogen that is capable of causing a deleterious effect, either alone or in combination with another factor, such as a second biological contaminant or pathogen or a native protein (wild-type or mutant) or antibody, in the biological material and/or a recipient thereof.

As used herein, the term “blood components” is intended to mean one or more of the components that may be separated from whole blood and include, but are not limited to, the following: cellular blood components, such as red blood cells, white blood cells, and platelets; blood proteins, such as blood clotting factors, enzymes, albumin, plasminogen, fibrinogen, and immunoglobulins; and liquid blood components, such as plasma, plasma protein fraction (PPF), cryoprecipitate, plasma fractions, and plasma-containing compositions.

As used herein, the term “cellular blood component” is intended to mean one or more of the components of whole blood that comprises cells, such as red blood cells, white blood cells, stem cells, and platelets.

As used herein, the term “blood protein” is intended to mean one or more of the proteins that are normally found in whole blood. Illustrative examples of blood proteins found in mammals, including humans, include, but are not limited to, the following: coagulation proteins, both vitamin K-dependent, such as Factor VII and Factor IX, and non-vitamin K-dependent, such as Factor VIII and von Willebrands factor; albumin; lipoproteins, including high density lipoproteins (HDL), low density lipoproteins (LDL), and very low density lipoproteins (VLDL); complement proteins; globulins, such as immunoglobulins IgA, IgM, IgG and IgE; and the like. A preferred group of blood proteins includes Factor I (fibrinogen), Factor II (prothrombin), Factor III (tissue factor), Factor V (proaccelerin), Factor VI (accelerin), Factor VII (proconvertin, serum prothrombin conversion), Factor VIII (antihemophiliac factor A), Factor IX (antihemophiliac factor B), Factor X (Stuart-Prower factor), Factor XI (plasma thromboplastin antecedent), Factor XII (Hageman factor), Factor XIII (protransglutamidase), von Willebrands factor (vWF), Factor Ia, Factor IIa, Factor IIIa, Factor Va, Factor VIa, Factor VIIa, Factor VIIIa, Factor IXa, Factor Xa, Factor XIa, Factor XIIa, and Factor XIIIa. Another preferred group of blood proteins includes proteins found inside red blood cells, such as hemoglobin and various growth factors, and derivatives of these proteins.

As used herein, the term “liquid blood component” is intended to mean one or more of the fluid, non-cellular components of whole blood, such as plasma (the fluid, non-cellular portion of the whole blood of humans or animals as found prior to coagulation) and serum (the fluid, non-cellular portion of the whole blood of humans or animals as found after coagulation).

As used herein, the term “a biologically compatible solution” is intended to mean a solution to which a biological material may be exposed, such as by being suspended or dissolved therein, and remain viable, i.e., retain its essential biological, pharmacological, and physiological characteristics.

As used herein, the term “a biologically compatible buffered solution” is intended to mean a biologically compatible solution having a pH and osmotic properties (e.g., tonicity, osmolality, and/or oncotic pressure) suitable for maintaining the integrity of the material(s) therein, including suitable for maintaining essential biological, pharmacological, and physiological characteristics of the material(s) therein. Suitable biologically compatible buffered solutions typically have a pH between about 2 and about 8.5, and are isotonic or only moderately hypotonic or hypertonic. Biologically compatible buffered solutions are known and readily available to those of skill in the art.

As used herein, the term “stabilizer mixture” is intended to mean the combination of two or more compounds or materials that, alone and/or in combination, reduce damage to the biological material being irradiated to a level that is insufficient to preclude the safe and effective use of the material. Illustrative examples of stabilizers that are suitable for use in a stabilizer mixture include, but are not limited to, the following, including structural analogs and derivatives thereof: antioxidants; free radical scavengers, including spin traps, such as tert-butyl-nitrosobutane (tNB), a-phenyl-tert-butylnitrone (PBN), 5,5-dimethylpyrroline-N-oxide (DMPO), tert-butylnitrosobenzene (BNB), a-(4-pyridyl-1-oxide)-N-tert-butylnitrone (4-POBN) and 3,5-dibromo-4-nitroso-benzenesulphonic acid (DBNBS); combination stabilizers, i.e., stabilizers which are effective at quenching both Type I and Type II photodynamic reactions; and ligands, ligand analogs, substrates, substrate analogs, modulators, modulator analogs, stereoisomers, inhibitors, and inhibitor analogs, such as heparin, that stabilize the molecule(s) to which they bind. Preferred examples of additional stabilizers include, but are not limited to, the following: fatty acids, including 6,8-dimercapto-octanoic acid (lipoic acid) and its derivatives and analogues (alpha, beta, dihydro, bisno and tetranor lipoic acid), thioctic acid, 6,8-dimercapto-octanoic acid, dihydrolopoate (DL-6,8-dithioloctanoic acid methyl ester), lipoamide, bisonor methyl ester and tetranor-dihydrolipoic acid, omega-3 fatty acids, omega-6 fatty acids, omega-9 fatty acids, furan fatty acids, oleic, linoleic, linolenic, arachidonic, eicosapentaenoic (EPA), docosahexaenoic (DHA), and palmitic acids and their salts and derivatives; carotenes, including alpha-, beta-, and gamma-carotenes; Co-Q10; xanthophylls; sucrose, polyhydric alcohols, such as glycerol, mannitol, inositol, and sorbitol; sugars, including derivatives and stereoisomers thereof, such as xylose, glucose, ribose, mannose, fructose, erythrose, threose, idose, arabinose, lyxose, galactose, allose, altrose, gulose, talose, and trehalose; amino acids and derivatives thereof, including both D- and L-forms and mixtures thereof, such as arginine, lysine, alanine, valine, leucine, isoleucine, proline, phenylalanine, glycine, serine, threonine, tyrosine, asparagine, glutamine, aspartic acid, histidine, N-acetylcysteine (NAC), glutamic acid, tryptophan, sodium capryl N-acetyl tryptophan, and methionine; azides, such as sodium azide; enzymes, such as Superoxide Dismutase (SOD), Catalase, and 4, 5 and 6 desaturases; uric acid and its derivatives, such as 1,3-dimethyluric acid and dimethylthiourea; allopurinol; thiols, such as glutathione and reduced glutathione and cysteine; trace elements, such as selenium, chromium, and boron; vitamins, including their precursors and derivatives, such as vitamin A, vitamin C (including its derivatives and salts such as sodium ascorbate and palmitoyl ascorbic acid) and vitamin E (and its derivatives and salts such as alpha-, beta-, gamma-, delta-, epsilon-, zeta-, and eta-tocopherols, tocopherol acetate and alpha-tocotrienol); chromanol-alpha-C6; 6-hydroxy-2,5,7,8-tetramethylchroma-2 carboxylic acid (Trolox) and derivatives; extraneous proteins, such as gelatin and albumin; tris-3-methyl-1-phenyl-2-pyrazolin-5-one (MCI-186); citiolone; puercetin; chrysin; dimethyl sulfoxide (DMSO); piperazine diethanesulfonic acid (PIPES); imidazole; methoxypsoralen (MOPS); 1,2-dithiane-4,5-diol; reducing substances, such as butylated hydroxyanisole (BHA) and butylated hydroxytoluene (BHT); cholesterol, including derivatives and its various oxidized and reduced forms thereof, such as low density lipoprotein (LDL), high density lipoprotein (HDL), and very low density lipoprotein (VLDL); probucol; indole derivatives; thimerosal; lazaroid and tirilazad mesylate; proanthenols; proanthocyanidins; ammonium sulfate; Pegorgotein (PEG-SOD); N-tert-butyl-alpha-phenylnitrone (PBN); 4-hydroxy-2,2,6,6-tetramethylpiperidin-1-oxyl (Tempol); mixtures of ascorbate, urate and Trolox C (Asc/urate/Trolox C); proteins, such as albumin, and peptides of two or more amino acids, any of which may be either naturally occurring amino acids, i.e., L-amino acids, or non-naturally occurring amino acids, i.e., D-amino acids, and mixtures, derivatives, and analogs thereof, including, but are not limited to, arginine, lysine, alanine, valine, leucine, isoleucine, proline, phenylalanine, glycine, histidine, glutamic acid, tryptophan (Trp), serine, threonine, tyrosine, asparagine, glutamine, aspartic acid, cysteine, methionine, and derivatives thereof, such as N-acetylcysteine (NAC) and sodium capryl N-acetyl tryptophan, as well as homologous dipeptide stabilizers (composed of two identical amino acids), including such naturally occurring amino acids, as Gly-Gly (glycylglycine) and Trp-Trp, and heterologous dipeptide stabilizers (composed of different amino acids), such as carnosine (b-alanyl-histidine), anserine (b-alanyl-methylhistidine), and Gly-Trp; and flavonoids/flavonols, such as quercetin, rutin, silybin, silidianin, silicristin, silymarin, apigenin, apiin, chrysin, morin, isoflavone, flavoxate, gossypetin, myricetin, biacalein, kaempferol, curcumin, proanthocyanidin B2-3-O-gallate, epicatechin gallate, epigallocatechin gallate, epigallocatechin, gallic acid, epicatechin, dihydroquercetin, quercetin chalcone, 4,4′-dihydroxy-chalcone, isoliquiritigenin, phloretin, coumestrol, 4′,7-dihydroxy-flavanone, 4′,5-dihydroxy-flavone, 4′,6-dihydroxy-flavone, luteolin, galangin, equol, biochanin A, daidzein, formononetin, genistein, amentoflavone, bilobetin, taxifolin, delphinidin, malvidin, petunidin, pelargonidin, malonylapiin, pinosylvin, 3-methoxyapigenin, leucodelphinidin, dihydrokaempferol, apigenin 7-O-glucoside, pycnogenol, aminoflavone, purpurogallin fisetin, 2′,3′-dihydroxylfavone, 3-hydroxyflavone, 3′,4′-dihydroxyflavone, catechin, 7-flavonoxyacetic acid ethyl ester, catechin, hesperidin, and naringin. Particularly preferred examples include single stabilizers or combinations of stabilizers that are effective at quenching both Type I and Type II photodynamic reactions, and volatile stabilizers, which can be applied as a gas and/or easily removed by evaporation, low pressure, and similar methods.

As used herein, the term “residual solvent content” is intended to mean the amount or proportion of freely-available liquid in the biological material. Freely-available liquid means the liquid, such as water or an organic solvent (e.g., ethanol, isopropanol, polyethylene glycol, etc.), present in the biological material being sterilized that is not bound to or complexed with one or more of the non-liquid components of the biological material. Freely-available liquid includes intracellular water. The residual solvent contents related as water referenced herein refer to levels determined by the FDA approved, modified Karl Fischer method (Meyer and Boyd, Analytical Chem., 31:215-219, 1959; May, et al., J. Biol. Standardization, 10:249-259, 1982; Centers for Biologics Evaluation and Research, FDA, Docket No. 89D-0140, 83-93; 1990) or by near infrared spectroscopy. Quantitation of the residual levels of other solvents may be determined by means well known in the art, depending upon which solvent is employed. The proportion of residual solvent to solute may also be considered to be a reflection of the concentration of the solute within the solvent. When so expressed, the greater the concentration of the solute, the lower the amount of residual solvent.

As used herein, the term “sensitizer” is intended to mean a substance that selectively targets viruses, bacteria (including inter- and intracellular bacteria, such as mycoplasmas, ureaplasmas, nanobacteria, chlamydia, rickettsias), yeasts, molds, fungi, single or multicellular parasites, and/or prions or similar agents responsible, alone or in combination, for TSEs, rendering them more sensitive to inactivation by radiation, therefore permitting the use of a lower rate or dose of radiation and/or a shorter time of irradiation than in the absence of the sensitizer. Illustrative examples of suitable sensitizers include, but are not limited to, the following: psoralen and its derivatives and analogs (including 3-carboethoxy psoralens); inactines and their derivatives and analogs; angelicins, khellins and coumarins which contain a halogen substituent and a water solubilization moiety, such as quaternary ammonium ion or phosphonium ion; nucleic acid binding compounds; brominated hematoporphyrin; phthalocyanines; purpurins; porphyrins; halogenated or metal atom-substituted derivatives of dihematoporphyrin esters, hematoporphyrin derivatives, benzoporphyrin derivatives, hydrodibenzoporphyrin dimaleimade, hydrodibenzoporphyrin, dicyano disulfone, tetracarbethoxy hydrodibenzoporphyrin, and tetracarbethoxy hydrodibenzoporphyrin dipropionamide; doxorubicin and daunomycin, which may be modified with halogens or metal atoms; netropsin; BD peptide, S2 peptide; S-303 (ALE compound); dyes, such as hypericin, methylene blue, eosin, fluoresceins (and their derivatives), flavins, merocyanine 540; photoactive compounds, such as bergapten; and SE peptide. In addition, atoms which bind to prions, and thereby increase their sensitivity to inactivation by radiation, may also be used. An illustrative example of such an atom would be the Copper ion, which binds to the prion protein and, with a Z number higher than the other atoms in the protein, increases the probability that the prion protein will absorb energy during irradiation, particularly gamma irradiation.

As used herein, the term “proteinaceous material” is intended to mean any material derived or obtained from a living organism that comprises at least one protein or peptide. A proteinaceous material may be a naturally occurring material, either in its native state or following processing/purification and/or derivatization, or an artificially produced material, produced by chemical synthesis or recombinant/transgenic technology and, optionally, process/purified and/or derivatized. Illustrative examples of proteinaceous materials include, but are not limited to, the following: proteins and peptides produced from cell culture; milk and other dairy products; ascites; hormones; growth factors; materials, including pharmaceuticals, extracted or isolated from animal tissue or plant matter, such as heparin, insulin, and inulin; plasma, including fresh, frozen and freeze-dried, and plasma protein fraction; fibrinogen and derivatives thereof, fibrin, fibrin I, fibrin II, soluble fibrin and fibrin monomer, and/or fibrin sealant products; whole blood; protein C; protein S; alpha-1 anti-trypsin (alpha-1 protease inhibitor); butyl-cholinesterase; anticoagulants, such as coumarin drugs (warfarin); streptokinase; tissue plasminogen activator (tPA); erythropoietin (EPO); urokinase; Neupogen™; anti-thrombin-3; alpha-galactosidase; iduronate-2-sulfatase; (fetal) bovine serum/horse serum; meat; immunoglobulins, including anti-sera, monoclonal antibodies, polyclonal antibodies, and genetically engineered or produced antibodies; albumin; alpha-globulins; beta-globulins; gamma-globulins; coagulation proteins; complement proteins; and interferons.

As used herein, the term “radiation” is intended to mean radiation of sufficient energy to sterilize at least some component of the irradiated biological material. Types of radiation include, but are not limited to, the following: (i) corpuscular (streams of subatomic particles such as neutrons, electrons, and/or protons); (ii) electromagnetic (originating in a varying electromagnetic field, such as radio waves, visible (both mono and polychromatic) and invisible light, infrared, ultraviolet radiation, x-radiation, and gamma rays and mixtures thereof); and (iii) sound and pressure waves. Such radiation is often described as either ionizing (capable of producing ions in irradiated materials) radiation, such as gamma rays, and non-ionizing radiation, such as visible light. The sources of such radiation may vary and, in general, the selection of a specific source of radiation is not critical provided that sufficient radiation is given in an appropriate time and at an appropriate rate to effect sterilization. In practice, gamma radiation is usually produced by isotopes of Cobalt or Cesium, while UV and X-rays are produced by machines that emit UV and X-radiation, respectively, and electrons are often used to sterilize materials in a method known as “E-beam” irradiation that involves their production via a machine. Visible light, both mono- and polychromatic, is produced by machines and may, in practice, be combined with invisible light, such as infrared and UV, that is produced by the same machine or a different machine.

As used herein, the term “to protect” is intended to mean to reduce any damage to the biological material being irradiated, that would otherwise result from the irradiation of that material, to a level that is insufficient to preclude the safe and effective use of the material following irradiation. In other words, a substance or process “protects” a biological material from radiation if the presence of that substance or carrying out that process results in less damage to the material from irradiation than in the absence of that substance or process. Thus, a biological material may be used safely and effectively after irradiation in the presence of a substance or following performance of a process that “protects” the material, but could not be used safely and effectively after irradiation under identical conditions but in the absence of that substance or the performance of that process.

As used herein, an “acceptable level” of damage may vary depending upon certain features of the particular method(s) of the present invention being employed, such as the nature and characteristics of the particular biological material and/or non-aqueous solvent(s) being used, and/or the intended use of the biological material being irradiated, and can be determined empirically by one skilled in the art. An “unacceptable level” of damage would therefore be a level of damage that would preclude the safe and effective use of the biological material being sterilized. The particular level of damage in a given biological material may be determined using any of the methods and techniques known to one skilled in the art.

B. Particularly Preferred Embodiments

A first preferred embodiment of the present invention is directed to a method for sterilizing a biological material that is sensitive to radiation comprising: (i) adding to a biological material at least one stabilizer mixture in an amount effective to protect the biological material from radiation; and (ii) irradiating the biological material with radiation at an effective rate for a time effective to sterilize the biological material.

A second preferred embodiment of the present invention is directed to a method for sterilizing a biological material that is sensitive to radiation comprising: (i) reducing the residual solvent content of a biological material; (ii) adding to the biological material at least one stabilizer mixture; and (iii) irradiating the biological material with radiation at an effective rate for a time effective to sterilize the biological material, wherein the level of residual solvent content and the amount of stabilizer mixture are together effective to protect the biological material from radiation. The order of steps (i) and (ii) may, of course, be reversed as desired.

A third preferred embodiment of the present invention is directed to a method for sterilizing a biological material that is sensitive to radiation comprising: (i) reducing the temperature of a biological material; (ii) adding to the biological material at least one stabilizer mixture; and (iii) irradiating the biological material with radiation at an effective rate for a time effective to sterilize the biological material, wherein the temperature and the amount of stabilizer mixture are together effective to protect the biological material from radiation. The order of steps (i) and (ii) may, of course, be reversed as desired.

A fourth preferred embodiment of the present invention is directed to a method for sterilizing a biological material that is sensitive to radiation comprising: (i) reducing the residual solvent content of a biological material; (ii) adding to the biological material at least one stabilizer mixture; (iii) reducing the temperature of the biological material; and (iv) irradiating the biological material with radiation at an effective rate for a time effective to sterilize the biological material, wherein the temperature and the amount of stabilizer mixture are together effective to protect the biological material from radiation. According to this embodiment, steps (i) (ii) and (iii) may be performed in any order.

According to the methods of the present invention, a stabilizer mixture is added prior to irradiation of the biological material with radiation. This stabilizer mixture is preferably added to the biological material in an amount that is effective to protect the biological material from the radiation. Suitable amounts of stabilizer mixture may vary depending upon certain features of the particular method(s) of the present invention being employed, such as the particular stabilizer mixture being used and/or the nature and characteristics of the particular biological material being irradiated and/or its intended use, and can be determined empirically by one skilled in the art.

According to certain methods of the present invention, the residual solvent content of the biological material is reduced prior to irradiation of the biological material with radiation. The residual solvent content is preferably reduced to a level that is effective to protect the biological material from the radiation. Suitable levels of residual solvent content may vary depending upon certain features of the particular method(s) of the present invention being employed, such as the nature and characteristics of the particular biological material being irradiated and/or its intended use, and can be determined empirically by one skilled in the art. There may be biological materials for which it is desirable to maintain the residual solvent content to within a particular range, rather than a specific value.

When the solvent is water, and particularly when the biological material is in a solid phase, the residual solvent content is generally less than about 15%, typically less than about 10%, more typically less than about 9%, even more typically less than about 8%, usually less than about 5%, preferably less than about 3.0%, more preferably less than about 2.0%, even more preferably less than about 1.0%, still more preferably less than about 0.5%, still even more preferably less than about 0.2% and most preferably less than about 0.08%.

The solvent may preferably be a non-aqueous solvent, more preferably a non-aqueous solvent that is not prone to the formation of free-radicals upon irradiation, and most preferably a non-aqueous solvent that is not prone to the formation of free-radicals upon irradiation and that has little or no dissolved oxygen or other gas(es) that is (are) prone to the formation of free-radicals upon irradiation. Volatile non-aqueous solvents are particularly preferred, even more particularly preferred are non-aqueous solvents that are stabilizers, such as ethanol and acetone.

In certain embodiments of the present invention, the solvent may be a mixture of water and a non-aqueous solvent or solvents, such as ethanol and/or acetone. In such embodiments, the non-aqueous solvent(s) is preferably a non-aqueous solvent that is not prone to the formation of free-radicals upon irradiation, and most preferably a non-aqueous solvent that is not prone to the formation of free-radicals upon irradiation and that has little or no dissolved oxygen or other gas(es) that is (are) prone to the formation of free-radicals upon irradiation. Volatile non-aqueous solvents are particularly preferred, even more particularly preferred are non-aqueous solvents that are stabilizers, such as ethanol and acetone.

In a preferred embodiment, when the residual solvent is water, the residual solvent content of a biological material is reduced by dissolving or suspending the biological material in a non-aqueous solvent that is capable of dissolving water. Preferably, such a non-aqueous solvent is not prone to the formation of free-radicals upon irradiation and has little or no dissolved oxygen or other gas(es) that is (are) prone to the formation of free-radicals upon irradiation.

When the biological material is in a liquid phase, reducing the residual solvent content may be accomplished by any of a number of means, such as by increasing the solute concentration. In this manner, the concentration of protein in the biological material dissolved within the solvent may be increased to generally at least about 0.5%, typically at least about 1%, usually at least about 5%, preferably at least about 10%, more preferably at least about 15%, even more preferably at least about 20%, still even more preferably at least about 25%, and most preferably at least about 50%.

In certain embodiments of the present invention, the residual solvent content of a particular biological material may be found to lie within a range, rather than at a specific point. Such a range for the preferred residual solvent content of a particular biological material may be determined empirically by one skilled in the art.

While not wishing to be bound by any theory of operability, it is believed that the reduction in residual solvent content reduces the degrees of freedom of the biological material, reduces the number of targets for free radical generation and may restrict the solubility of these free radicals. Similar results might therefore be achieved by lowering the temperature of the biological material below its eutectic point or below its freezing point, or by vitrification to likewise reduce the degrees of freedom of the biological material. These results may permit the use of a higher rate and/or dose of radiation than might otherwise be acceptable. Thus, the methods described herein may be performed at any temperature that doesn't result in unacceptable damage to the biological material, i.e., damage that would preclude the safe and effective use of the biological material. Preferably, the methods described herein are performed at ambient temperature or below ambient temperature, such as below the eutectic point or freezing point of the biological material being irradiated.

The residual solvent content of the biological material may be reduced by any of the methods and techniques known to those skilled in the art for reducing solvent from a biological material without producing an unacceptable level of damage to the biological material. Preferred examples of such methods include, but are not limited to, lyophilization, evaporation, concentration, centrifugal concentration, vitrification and spray-drying.

A particularly preferred method for reducing the residual solvent content of a biological material is lyophilization.

Another particularly preferred method for reducing the residual solvent content of a biological material is spray-drying.

Another particularly preferred method for reducing the residual solvent content of a biological material is vitrification, which may be accomplished by any of the methods and techniques known to those skilled in the art, including the addition of solute and or additional solutes, such as sucrose, to raise the eutectic point of the biological material, followed by a gradual application of reduced pressure to the biological material in order to remove the residual solvent, such as water. The resulting glassy material will then have a reduced residual solvent content.

According to certain methods of the present invention, the biological material to be sterilized may be immobilized upon a solid surface by any means known and available to one skilled in the art. For example, the biological material to be sterilized may be present as a coating or surface on a biological or non-biological substrate.

The radiation employed in the methods of the present invention may be any radiation effective for the sterilization of the biological material being treated. The radiation may be corpuscular, including E-beam radiation. Preferably the radiation is electromagnetic radiation, including x-rays, infrared, visible light, UV light and mixtures of various wavelengths of electromagnetic radiation. A particularly preferred form of radiation is gamma radiation.

According to the methods of the present invention, the biological material is irradiated with the radiation at a rate effective for the sterilization of the biological material, while not producing an unacceptable level of damage to that material. Suitable rates of irradiation may vary depending upon certain features of the methods of the present invention being employed, such as the nature and characteristics of the particular biological material being irradiated, the particular form of radiation involved and/or the particular biological contaminants or pathogens being inactivated. Suitable rates of irradiation can be determined empirically by one skilled in the art. Preferably, the rate of irradiation is constant for the duration of the sterilization procedure. When this is impractical or otherwise not desired, a variable or discontinuous irradiation may be utilized.

According to the methods of the present invention, the rate of irradiation may be optimized to produce the most advantageous combination of product recovery and time required to complete the operation. Both low (<3 kGy/hour) and high (>3 kGy/hour) rates may be utilized in the methods described herein to achieve such results. The rate of irradiation is preferably be selected to optimize the recovery of the biological material while still sterilizing the biological material. Although reducing the rate of irradiation may serve to decrease damage to the biological material, it will also result in longer irradiation times being required to achieve a particular desired total dose. A higher dose rate may therefore be preferred in certain circumstances, such as to minimize logistical issues and costs, and may be possible when used in accordance with the methods described herein for protecting a biological material from irradiation.

According to a particularly preferred embodiment of the present invention, the rate of irradiation is not more than about 3.0 kGy/hour, more preferably between about 0.1 kGy/hr and 3.0 kGy/hr, even more preferably between about 0.25 kGy/hr and 2.0 kGy/hour, still even more preferably between about 0.5 kGy/hr and 1.5 kGy/hr and most preferably between about 0.5 kGy/hr and 1.0 kGy/hr.

According to another particularly preferred embodiment of the present invention, the rate of irradiation is at least about 3.0 kGy/hr, more preferably at least about 6 kGy/hr, even more preferably at least about 16 kGy/hr, and even more preferably at least about 30 kGy/hr and most preferably at least about 45 kGy/hr or greater.

According to another particularly preferred embodiment of the present invention, the maximum acceptable rate of irradiation is inversely proportional to the molecular mass of the biological material being irradiated.

According to the methods of the present invention, the biological material to be sterilized is irradiated with the radiation for a time effective for the sterilization of the biological material. Combined with irradiation rate, the appropriate irradiation time results in the appropriate dose of irradiation being applied to the biological material. Suitable irradiation times may vary depending upon the particular form and rate of radiation involved and/or the nature and characteristics of the particular biological material being irradiated. Suitable irradiation times can be determined empirically by one skilled in the art.

According to the methods of the present invention, the biological material to be sterilized is irradiated with radiation up to a total dose effective for the sterilization of the biological material, while not producing an unacceptable level of damage to that material. Suitable total doses of radiation may vary depending upon certain features of the methods of the present invention being employed, such as the nature and characteristics of the particular biological material being irradiated, the particular form of radiation involved and/or the particular biological contaminants or pathogens being inactivated. Suitable total doses of radiation can be determined empirically by one skilled in the art. Preferably, the total dose of radiation is at least 25 kGy, more preferably at least 45 kGy, even more preferably at least 75 kGy, and still more preferably at least 100 kGy or greater, such as 150 kGy or 200 kGy or greater.

The particular geometry of the biological material being irradiated, such as the thickness and distance from the source of radiation, may be determined empirically by one skilled in the art. A preferred embodiment is a geometry that provides for an even rate of irradiation throughout the material. A particularly preferred embodiment is a geometry that results in a short path length for the radiation through the material, thus minimizing the differences in radiation dose between the front and back of the material. This may be further minimized in some preferred geometries, particularly those wherein the material has a constant radius about its axis that is perpendicular to the radiation source, by the utilization of a means of rotating the preparation about said axis.

Similarly, according to certain methods of the present invention, an effective package for containing the biological material during irradiation is one which combines stability under the influence of irradiation, and which minimizes the interactions between the package and the radiation. Preferred packages maintain a seal against the external environment before, during and post-irradiation, and are not reactive with the biological material within, nor do they produce chemicals that may interact with the material within. Particularly preferred examples include but are not limited to containers that comprise glasses stable when irradiated, stoppered with stoppers made of rubber that is relatively stable during radiation and liberates a minimal amount of compounds from within, and sealed with metal crimp seals of aluminum or other suitable materials with relatively low Z numbers. Suitable materials can be determined by measuring their physical performance, and the amount and type of reactive leachable compounds post-irradiation and by examining other characteristics known to be important to the containment of biological materials empirically by one skilled in the art.

According to certain methods of the present invention, an effective amount of at least one sensitizing compound may optionally be added to the biological material prior to irradiation, for example to enhance the effect of the irradiation on the biological contaminant(s) or pathogen(s) therein, while employing the methods described herein to minimize the deleterious effects of irradiation upon the biological material. Suitable sensitizers are known to those skilled in the art, and include psoralens and their derivatives and inactines and their derivatives.

According to the methods of the present invention, the irradiation of the biological material may occur at any temperature that is not deleterious to the biological material being sterilized. According to one preferred embodiment, the biological material is irradiated at ambient temperature. According to an alternate preferred embodiment, the biological material is irradiated at reduced temperature, i.e. a temperature below ambient temperature or lower, such as 0° C., −20° C., −40° C., −60° C., −78° C. or −196° C. According to this embodiment of the present invention, the biological material is preferably irradiated at or below the freezing or eutectic point of the biological material. According to another alternate preferred embodiment, the biological material is irradiated at elevated temperature, i.e. a temperature above ambient temperature or higher, such as 37° C., 60° C., 72° C. or 80° C. While not wishing to be bound by any theory, the use of elevated temperature may enhance the effect of irradiation on the biological contaminant(s) or pathogen(s) and therefore allow the use of a lower total dose of radiation.

Most preferably, the irradiation of the biological material occurs at a temperature that protects the material from radiation. Suitable temperatures can be determined empirically by one skilled in the art.

In certain embodiments of the present invention, the temperature at which irradiation is performed may be found to lie within a range, rather than at a specific point. Such a range for the preferred temperature for the irradiation of a particular biological material may be determined empirically by one skilled in the art.

According to the methods of the present invention, the irradiation of the biological material may occur at any pressure which is not deleterious to the biological material being sterilized. According to one preferred embodiment, the biological material is irradiated at elevated pressure. More preferably, the biological material is irradiated at elevated pressure due to the application of sound waves or the use of a volatile. While not wishing to be bound by any theory, the use of elevated pressure may enhance the effect of irradiation on the biological contaminant(s) or pathogen(s) and/or enhance the protection afforded by one or more stabilizers, and therefore allow the use of a lower total dose of radiation. Suitable pressures can be determined empirically by one skilled in the art.

Generally, according to the methods of the present invention, the pH of the biological material undergoing sterilization is about 7. In some embodiments of the present invention, however, the biological material may have a pH of less than 7, preferably less than or equal to 6, more preferably less than or equal to 5, even more preferably less than or equal to 4, and most preferably less than or equal to 3. In alternative embodiments of the present invention, the biological material may have a pH of greater than 7, preferably greater than or equal to 8, more preferably greater than or equal to 9, even more preferably greater than or equal to 10, and most preferably greater than or equal to 11. According to certain embodiments of the present invention, the pH of the material undergoing sterilization is at or near the isoelectric point(s) of one or more of the components of the biological material. Suitable pH levels can be determined empirically by one skilled in the art.

Similarly, according to the methods of the present invention, the irradiation of the biological material may occur under any atmosphere that is not deleterious to the biological material being treated. According to one preferred embodiment, the biological material is held in a low oxygen atmosphere or an inert atmosphere. When an inert atmosphere is employed, the atmosphere is preferably composed of a noble gas, such as helium or argon, more preferably a higher molecular weight noble gas, and most preferably argon. According to another preferred embodiment, the biological material is held under vacuum while being irradiated. According to a particularly preferred embodiment of the present invention, a biological material (lyophilized, liquid or frozen) is stored under vacuum or an inert atmosphere (preferably a noble gas, such as helium or argon, more preferably a higher molecular weight noble gas, and most preferably argon) prior to irradiation. According to an alternative preferred embodiment of the present invention, a liquid biological material is held under low pressure, to decrease the amount of gas, particularly oxygen, dissolved in the liquid, prior to irradiation, either with or without a prior step of solvent reduction, such as lyophilization. Such degassing may be performed using any of the methods known to one skilled in the art.

In another preferred embodiment, where the biological material contains oxygen or other gases dissolved within or associated with it, the amount of these gases within or associated with the material may be reduced by any of the methods and techniques known and available to those skilled in the art, such as the controlled reduction of pressure within a container (rigid or flexible) holding the material to be treated or by placing the material in a container of approximately equal volume.

In certain embodiments of the present invention, when the biological material to be treated is a tissue, the stabilizer mixture is introduced according to any of the methods and techniques known and available to one skilled in the art, including soaking the tissue in a solution containing the stabilizers, preferably under pressure, at elevated temperature and/or in the presence of a penetration enhancer, such as dimethylsulfoxide. Other methods of introducing the stabilizer mixture into a tissue include, but are not limited to, applying a gas containing the stabilizers, preferably under pressure and/or at elevated temperature, injection of the stabilizers or a solution containing the stabilizers directly into the tissue, placing the tissue under reduced pressure and then introducing a gas or solution containing the stabilizers, dehydration of the tissue by means known to those skilled in the art, followed by re-hydration using a solution containing said stabilizer(s), and followed after irradiation, when desired, by subsequent dehydration with or without an additional re-hydration in a solution or solutions without said stabilizer(s), and combinations of two or more of these methods. One or more sensitizers may also be introduced into a tissue according to such methods.

It will be appreciated that the combination of one or more of the features described herein may be employed to further minimize undesirable effects upon the biological material caused by irradiation, while maintaining adequate effectiveness of the irradiation process on the biological contaminant(s) or pathogen(s). For example, in addition to the use of a stabilizer mixture, a particular biological material may also be lyophilized, held at a reduced temperature and kept under vacuum prior to irradiation to further minimize undesirable effects.

The sensitivity of a particular biological contaminant or pathogen to radiation is commonly calculated by determining the dose necessary to inactivate or kill all but 37% of the agent in a sample, which is known as the D37 value. The desirable components of a biological material may also be considered to have a D37 value equal to the dose of radiation required to eliminate all but 37% of their desirable biological and physiological characteristics.

In accordance with certain preferred methods of the present invention, the sterilization of a biological material is conducted under conditions that result in a decrease in the D37 value of the biological contaminant or pathogen without a concomitant decrease in the D37 value of the biological material. In accordance with other preferred methods of the present invention, the sterilization of a biological material is conducted under conditions that result in an increase in the D37 value of the biological material. In accordance with the most preferred methods of the present invention, the sterilization of a biological material is conducted under conditions that result in a decrease in the D37 value of the biological contaminant or pathogen and a concomitant increase in the D37 value of the biological material.

### EXAMPLES

The following examples are illustrative, but not limiting, of the present invention. Other suitable modifications and adaptations are of the variety normally encountered by those skilled in the art and are fully within the spirit and scope of the present invention. Unless otherwise noted, all irradiation was accomplished using a 60Co source.

### Example 1

In this experiment, the protective effect of the combination of ascorbate (20 mM), urate (1.5 mM) and trolox (200 FM) on gamma irradiated freeze-dried anti-insulin monoclonal immunoglobulin supplemented with 1% bovine serum albumin (BSA) was evaluated.

Methods

Samples were freeze-dried for approximately 64 hours, stoppered under vacuum, and sealed with an aluminum, crimped seal. Samples were irradiated at a dose rate of 1.83-1.88 kGy/hr to a total dose of 45.1-46.2 kGy at 4° C.

Monoclonal immunoglobulin activity was determined by a standard ELISA protocol. Maxisorp plates were coated with human recombinant insulin at 2.5 μg/ml overnight at 4° C. The plate was blocked with 200 μl of blocking buffer (PBS, pH 7.4, 2% BSA) for two hours at 37° C., and then washed six times with wash buffer (TBS, pH 7, 0.05% TWEEN 20). Samples were re-suspended in 500 μl of high purity water (100 ng/μl), diluted to 5 μg/ml in a 300 μl U-bottomed plate coated for either overnight or for two hours with blocking buffer. Serial 3-fold dilutions were performed, with a final concentration of 0.0022 μg/ml. Plates were incubated for one hour at 37° C. with agitation, and then washed six times with a wash buffer. Phosphatase-labelled goat anti-mouse IgG (H+L) was diluted to 50 ng/ml in binding buffer, and 100 μl was added to each well. The plate was incubated for one hour at 37° C. with agitation, and washed six times with wash buffers. One hundred μl of Sigma-104 substrate (1 mg/ml in DEA buffer) was added to each well, and reacted at room temperature. The plate was read on a Multiskan MCC/340 at 405 nM with the 620 nM absorbance subtracted.

Results

Freeze-dried anti-insulin monoclonal immunoglobulin, supplemented with 1% BSA, and gamma irradiated to 45 kGy, retained only about 68% of potency. Samples irradiated to 45 kGy in the presence of the stabilizer mixture (ascorbate, urate and trolox), however, retained greater than 82% of potency.

### Example 2

In this experiment, the protective effect of the combination of 200: M Trolox, 1.5 mM urate, and 20 mM ascorbate on freeze-dried anti-insulin monoclonal immunoglobulin supplemented with 1% human serum albumin (HSA) and, optionally, 5% sucrose, irradiated at a high dose rate was evaluated.

Method

Samples were freeze-dried for approximately 64 hours, stoppered under vacuum, and sealed with an aluminum, crimped seal. Samples were irradiated at a dose rate of approximately 1.85 kGy/hr to a total dose of 45 kGy at 4° C.

Monoclonal immunoglobulin activity was determined by a standard ELISA protocol. Maxisorp plates were coated with human recombinant insulin at 2.5 μg/ml overnight at 4° C. The plate was blocked with 200 μl of blocking buffer (PBS, pH 7.4, 2% BSA) for two hours at 37° C., and then washed six times with wash buffer (TBS, pH 7, 0.05% TWEEN 20). Samples were re-suspended in 500 μl of high purity water (100 ng/μl), and diluted to 5 μg/ml in a 300 μl U-bottomed plate coated for either overnight or two hours with blocking buffer. Serial 3-fold dilutions were performed, with a final concentration of 0.0022 μg/ml. Plates were incubated for one hour at 37° C. with agitation, and then washed six times with wash buffer. Phosphatase-labelled goat anti-mouse IgG (H+L) was diluted to 50 ng/ml in binding buffer, and 100 μl was added to each well. The plate was incubated for one hour at 37° C. with agitation, and washed six times with wash buffers. One hundred μl of Sigma-104 substrate (1 mg/ml in DEA buffer) was added to each well and reacted at room temperature. The plate was read on a Multiskan MCC/340 at 405 nM with the 620 nM absorbance subtracted.

Results

Freeze-dried anti-insulin monoclonal immunoglobulin containing 1% HSA and the stabilizer mixture (trolox/urate/ascorbate) retained about 87% of activity following gamma irradiation to 45 kGy. Freeze-dried anti-insulin monoclonal immunoglobulin containing only 1% HSA retained only 67% of activity following gamma irradiation to 45 kGy.

Freeze-dried anti-insulin monoclonal immunoglobulin containing 1% HSA, 5% sucrose and the stabilizer mixture (trolox/urate/ascorbate) retained about 84% of activity following gamma irradiation to 45 kGy. Freeze-dried anti-insulin monoclonal immunoglobulin containing only 1% HSA and 5% sucrose retained only about 70% of activity following gamma irradiation to 45 kGy.

### Example 3

In this experiment, the protective effect of ascorbate (200 mM), alone or in combination with Gly-Gly (200 mM), on a liquid polyclonal antibody preparation was evaluated.

Method

In 2 ml glass vials, samples of IGIV (50 mg/ml) were prepared with either no stabilizer or the stabilizer of interest. Samples were irradiated with gamma radiation (45 kGy total dose, dose rate 1.8 kGy/hr, temperature 4° C.) and then assayed for functional activity and structural integrity.

Functional activity of independent duplicate samples was determined by measuring binding activity for rubella, mumps and CMV using the appropriate commercial enzyme immunoassay (EIA) kit obtained from Sigma, viz., the Rubella IgG EIA kit, the Mumps IgG EIA kit and the CMV IgG EIA kit.

Structural integrity was determined by gel filtration (elution buffer: 50 mM NaPi, 100 mM NaCl, pH 6.7; flow rate: 1 ml/min; injection volume 50 μl) and SDS-PAGE (pre-cast tris-glycine 4-20% gradient gel from Novex in a Hoefer Mighty Small Gel Electrophoresis Unit running at 125V; sample size: 10 μl).

Results

Functional Activity

Irradiation of liquid polyclonal antibody samples to 45 kGy resulted in the loss of approximately 1 log of activity for rubella (relative to unirradiated samples). The addition of ascorbate alone improved recovery, as did the addition of ascorbate in combination with the dipeptide Gly-Gly.

Similarly, irradiation of liquid polyclonal antibody samples to 45 kGy resulted in the loss of approximately 0.5-0.75 log of activity for mumps. The addition of ascorbate alone improved recovery, as did the addition of ascorbate in combination with the dipeptide Gly-Gly.

Likewise, irradiation of liquid polyclonal antibody samples to 45 kGy resulted in the loss of approximately 1 log of activity for CMV. The addition of ascorbate alone improved recovery, as did the addition of ascorbate in combination with the dipeptide Gly-Gly.

Structural Analysis

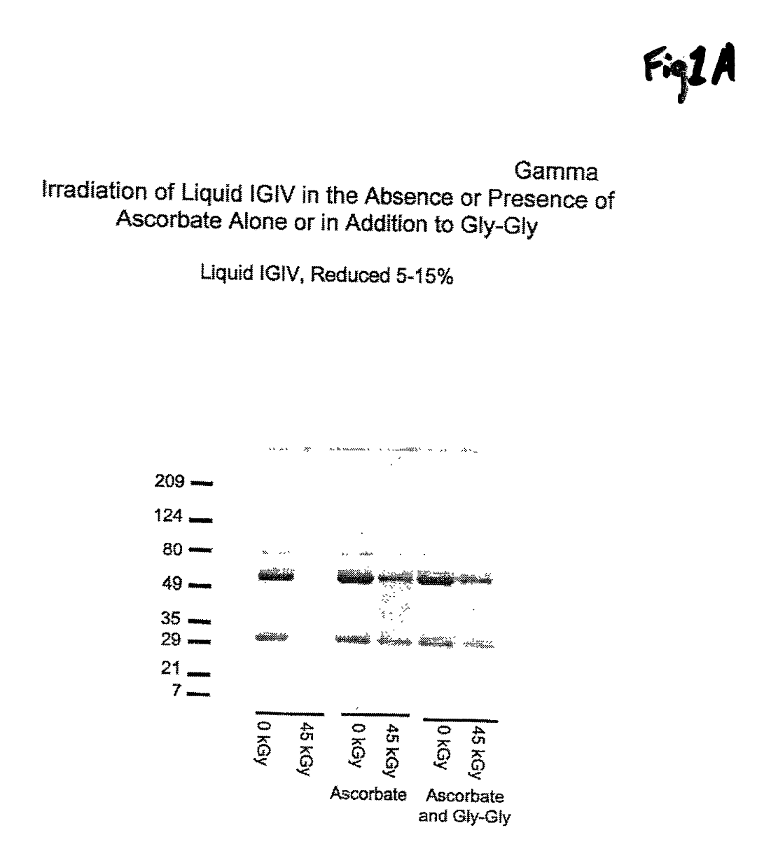

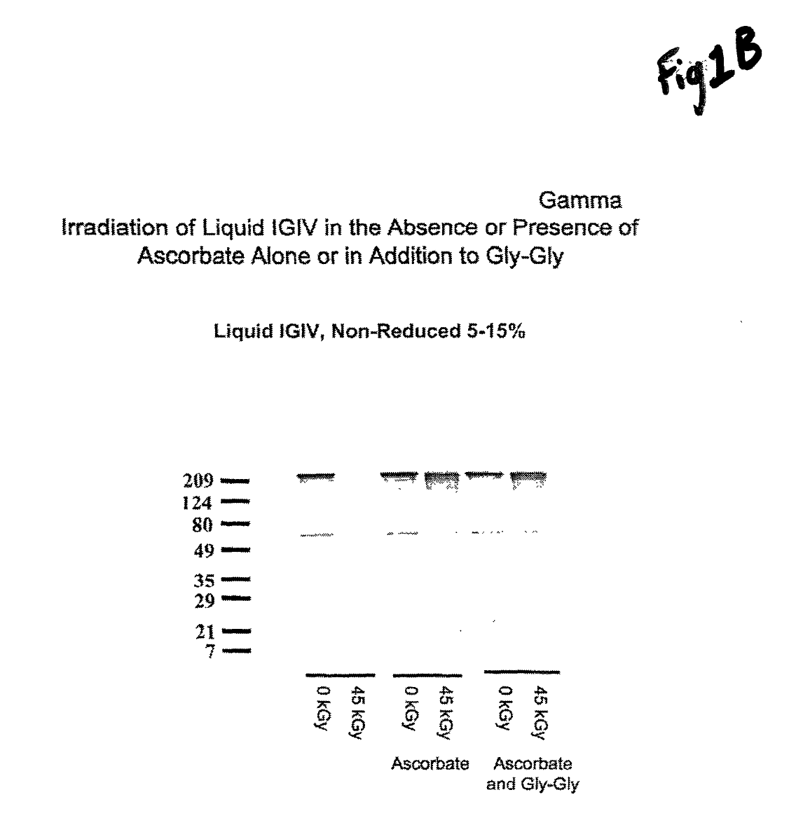

Liquid polyclonal antibody samples irradiated to 45 kGy in the absence of a stabilizer showed significant loss of material and evidence of both aggregation and fragmentation. The irradiated samples containing ascorbate or a combination of ascorbate and the dipeptide Gly-Gly exhibited only slight breakdown and some aggregation as demonstrated by gel filtration and SDS-PAGE (FIGS. 1A-1B).

### Example 4

In this experiment, the protective effect of ascorbate (20 mM) and/or Gly-Gly (20 mM) on lyophilized anti-insulin monoclonal immunoglobulin irradiated at a high dose rate was evaluated.

Method

Samples were freeze-dried for approximately 64 hours and stoppered under vacuum and sealed with an aluminum, crimped seal. Samples were irradiated at a dose rate of 30 kGy/hr to a total dose of 45 kGy at 4° C.

Monoclonal immunoglobulin activity was determined by a standard ELISA protocol. Maxisorp plates were coated with human recombinant insulin at 2.5 μg/ml overnight at 4° C. The plate was blocked with 200 μl of blocking buffer (PBS, pH 7.4, 2% BSA) for two hours at 37° C. and then washed six times with wash buffer (TBS, pH 7, 0.05% TWEEN 20). Samples were re-suspended in 500 μl of high purity water (100 ng/μl), diluted to 5 μg/ml in a 300 μl U-bottomed plate coated for either overnight or two hours with blocking buffer. Serial 3-fold dilutions were performed, with a final concentration of 0.0022 μg/ml. Plates were incubated for one hour at 37° C. with agitation and then washed six times with a wash buffer. Phosphatase-labelled goat anti-mouse IgG (H+L) was diluted to 50 ng/ml in binding buffer and 100 μl was added to each well. The plate was incubated for one hour at 37° C. with agitation and washed six times with wash buffers. One hundred μl of Sigma-104 substrate (1 mg/ml in DEA buffer) was added to each well and reacted at room temperature. The plate was read on a Multiskan MCC/340 at 405 nM with the 620 nM absorbance subtracted.

Results

Lyophilized anti-insulin monoclonal immunoglobulin gamma irradiated to 45 kGy resulted in an average loss in activity of ˜32% (average loss in avidity of ˜1.5 fold).

Lyophilized anti-insulin monoclonal immunoglobulin samples irradiated to 45 kGy in the presence of 20 mM ascorbate alone had a 15% loss in activity (˜1.1 fold loss in avidity), and those samples irradiated to 45 kGy in the presence of 20 mM Gly-Gly alone had a 23% loss in activity (˜1.3 fold loss in avidity).

In contrast, lyophilized anti-insulin monoclonal immunoglobulin samples irradiated to 45 kGy in the presence of the stabilizer mixture (20 mM ascorbate and 20 mM Gly-Gly) showed no loss in activity (no loss in avidity).

### Example 5

In this experiment, the protective effect of ascorbate (200 mM) and/or Gly-Gly (200 mM) on liquid anti-insulin monoclonal immunoglobulin irradiated to 45 kGy.

Method

Liquid samples containing 100 μg antibody (2 mg/ml) with 10% BSA were irradiated at a dose rate of 1.83-1.88 kGy/hr to a total dose of 45.1-46.2 kGy at 4° C.

Monoclonal immunoglobulin activity was determined by a standard ELISA protocol. Maxisorp plates were coated with human recombinant insulin at 2.5 μg/ml overnight at 4° C. The plate was blocked with 200 μl of blocking buffer (PBS, pH 7.4, 2% BSA) for two hours at 37° C. and then washed six times with wash buffer (TBS, pH 7, 0.05% TWEEN 20). Samples were re-suspended in 500 μl of high purity water (100 ng/μl), diluted to 5 μg/ml in a 300 μl U-bottomed plate coated for either overnight or two hours with blocking buffer. Serial 3-fold dilutions were performed, with a final concentration of 0.0022 μg/ml. Plates were incubated for one hour at 37° C. with agitation and then washed six times with a wash buffer. Phosphatase-labelled goat anti-mouse IgG (H+L) was diluted to 50 ng/ml in binding buffer and 100 μl was added to each well. The plate was incubated for one hour at 37° C. with agitation and washed six times with wash buffers. One hundred μl of Sigma-104 substrate (1 mg/ml in DEA buffer) was added to each well and reacted at room temperature. The plate was read on a Multiskan MCC/340 at 405 nM with the 620 nM absorbance subtracted.

Results

Liquid anti-insulin monoclonal immunoglobulin gamma irradiated to 45 kGy exhibited a complete loss of activity. Liquid anti-insulin monoclonal immunoglobulin samples irradiated to 45 kGy in the presence of 200 mM ascorbate alone exhibited a 48% loss in activity compared to unirradiated control.

In contrast, liquid anti-insulin monoclonal immunoglobulin samples irradiated to 45 kGy in the presence of the stabilizer mixture (200 mM ascorbate and 200 mM Gly-Gly) showed only a 29% loss in activity.

### Example 6

In this experiment, the protective effect of the combination of ascorbate (200 mM) and Gly-Gly (200 mM) on two different frozen enzyme preparations (a galactosidase and a sulfatase) was evaluated.

Method

In glass vials, 300 μl total volume containing 300 μg of enzyme (1 mg/ml) were prepared with either no stabilizer or the stabilizer of interest. Samples were irradiated with gamma radiation (45 kGy total dose, dose rate and temperature of either 1.616 kGy/hr and −21.5° C. or 5.35 kGy/hr and −21.9° C.) and then assayed for structural integrity.

Structural integrity was determined by SDS-PAGE. Three 12.5% gels were prepared according to the following recipe: 4.2 ml acrylamide; 2.5 ml 4×-Tris (pH 8.8); 3.3 ml water; 100 μl 10% APS solution; and 10 μl TEMED (tetramethylethylenediamine) and placed in an electrophoresis unit with 1×Running Buffer (15.1 g Tris base; 72.0 g glycine; 5.0 g SDS in 1 l water, diluted 5-fold). Irradiated and control samples (1 mg/ml) were diluted with Sample Buffer (+/−beta-mercaptoethanol) in Eppindorf tubes and then centrifuged for several minutes. 20 μl of each diluted sample (˜10 μg) were assayed.

Results

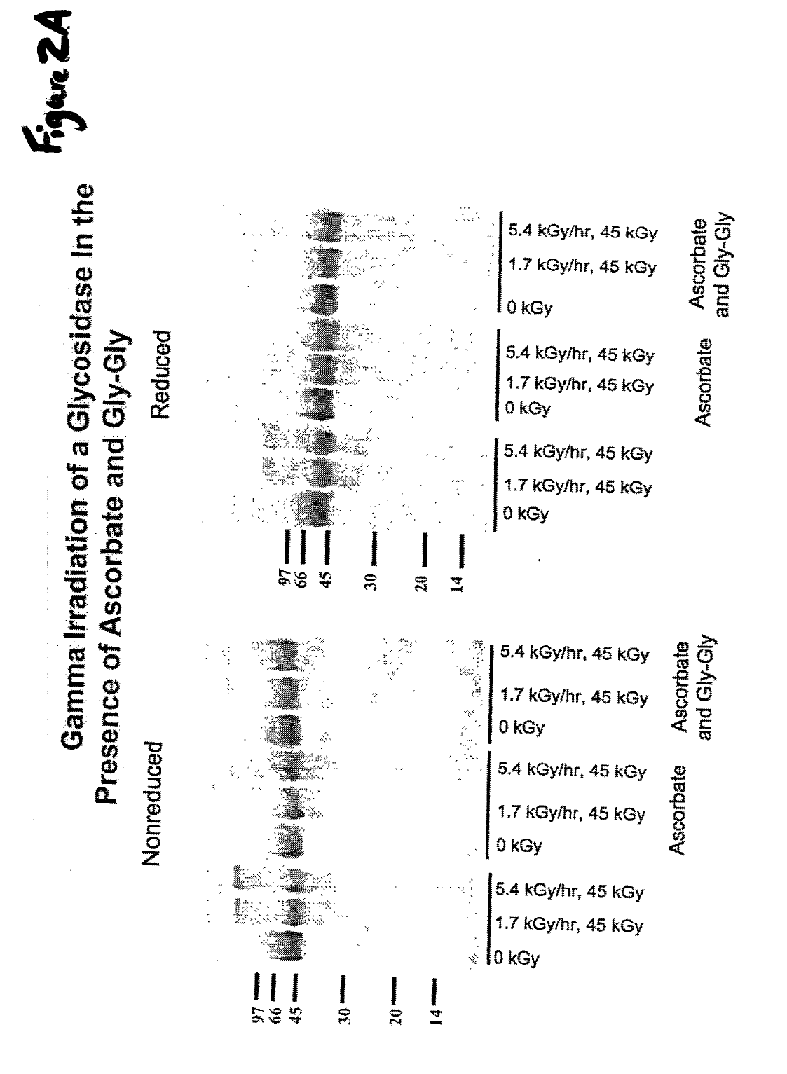

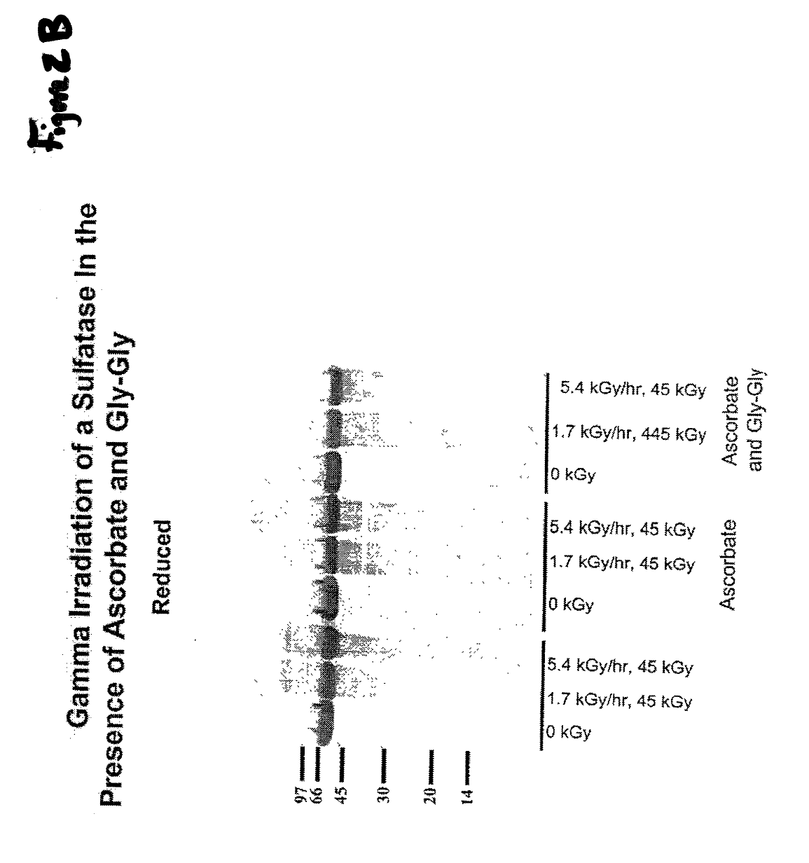

As shown in FIG. 2A, liquid galactosidase samples irradiated to 45 kGy in the absence of a stabilizer showed significant loss of material and evidence of both aggregation and fragmentation. Much greater recovery of material was obtained from the irradiated samples containing the combination of ascorbate and Gly-Gly.

As shown in FIG. 2B, liquid sulfatase samples irradiated to 45 kGy in the absence of a stabilizer showed significant loss of material and evidence of both aggregation and fragmentation. Much greater recovery of material was obtained from the irradiated samples containing the combination of ascorbate and Gly-Gly.

### Example 7

In this experiment, the protective effect of the combination of ascorbate (200 mM) and Gly-Gly (200 mM) on a frozen galactosidase preparation was evaluated.

Method

Samples were prepared in 2 ml glass vials containing 52.6 μl of a galactosidase solution (5.7 mg/ml), no stabilizer or the stabilizers of interest and sufficient water to make a total sample volume of 300 μl. Samples were irradiated at a dose rate of 1.616 or 5.35 kGy/hr at a temperature between −20 and −21.9° C. to a total dose of 45 kGy.

Structural integrity was determined by reverse phase chromatography. 10 μl of sample were diluted with 90 μl solvent A and then injected onto an Aquapore RP-300 (c-8) column (2.1×30 mm) mounted in an Applied Biosystems 130A Separation System Microbore HPLC. Solvent A: 0.1% trifluoroacetic acid; solvent B: 70% acetonitrile, 30% water, 0.085% trifluoroacetic acid.

Results

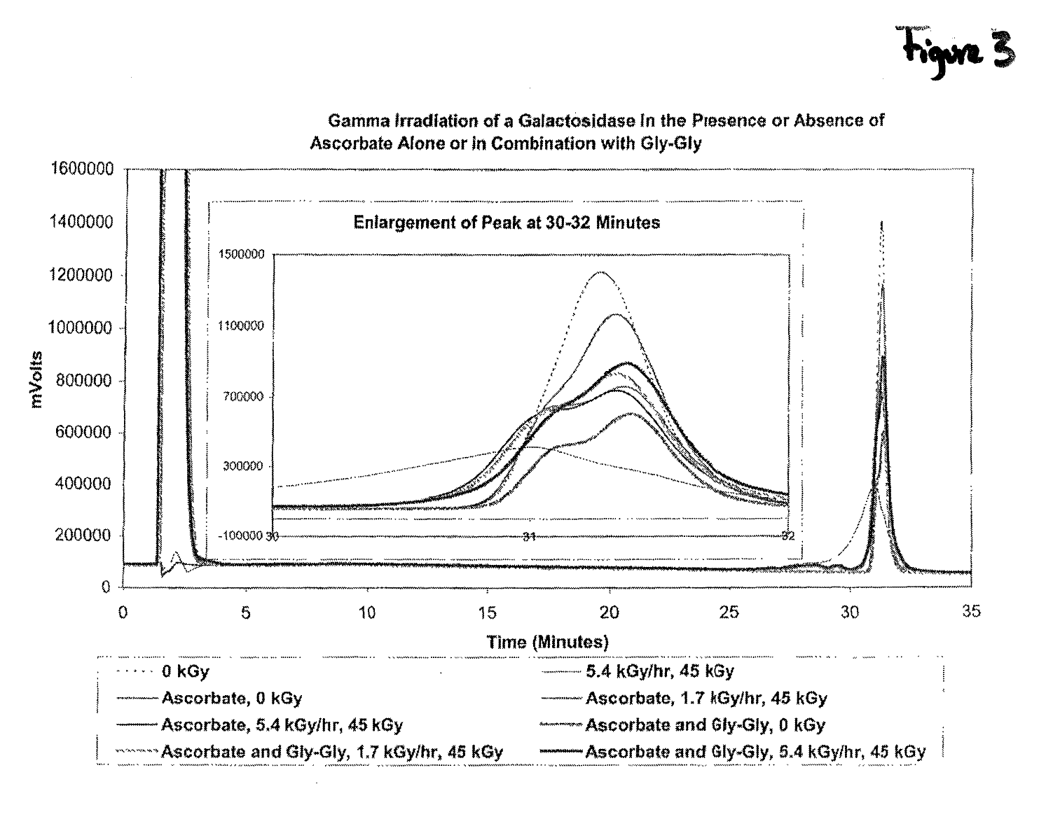

Liquid enzyme samples irradiated to 45 kGy in the absence of a stabilizer showed broadened and reduced peaks. As shown in FIG. 3, much greater recovery of material, as evidenced by significantly less reduction in peak size compared to control, was obtained from the irradiated samples containing the stabilizer mixture (ascorbate and Gly-Gly).

### Example 8

In this experiment, the protective effects of 200 mM glycylglycine, 200 mM ascorbate, and the combination of 200 mM Gly-Gly+200 mM ascorbate on gamma irradiated liquid anti-Ig Lambda Light Chain monoclonal antibody were evaluated.

Methods

Vials containing 33.8 μg of anti-Ig Lambda Light Chain monoclonal antibody (0.169 mg/mL) plus 200 mM Gly-Gly, 200 mM ascorbate, or the combination of 200 mM ascorbate and 200 mM Gly-Gly, were irradiated at a rate of 1.752 kGy/hr to a total dose of about 45 kGy at a temperature of 4° C.

ELISA assays were performed as follows. Two microtitre plates were coated with Human IgG1, Lambda Purified Myeloma Protein at 2 μg/ml, and stored overnight at 4EC. The next day, an ELISA technique was performed using the standard reagents used in the Anti-Insulin ELISA. Following a one hour block, a 10 μg/ml dilution of each sample set was added to the first column of the plate and then serially diluted 3-fold through column 12. Incubation was then performed for one hour at 37EC. Next, a 1:8,000 dilution was made of the secondary antibody, Phosphatase-Labeled Goat Anti-Mouse IgG was added, and incubation was performed for one hour at 37° C. Sigma 104-105 Phosphatase Substrate was added, color was allowed to develop for about 15 minutes, and the reaction was stopped by adding 0.5 M NaOH. Absorbance was measured at 405 nm-620 nm.

Results

Gamma irradiation of anti-Ig Lambda Light Chain monoclonal antibody to 45 kGy in the absence of stabilizers or in the presence of 200 mM Gly-Gly alone retained essentially no antibody activity. Samples that were gamma irradiated to 45 kGy in the presence of 200 mM ascorbate retained approximately 55% of antibody activity, but those irradiated in the presence of the stabilizer mixture (200 mM ascorbate and 200 mM Gly-Gly) retained approximately 86% of antibody activity.

### Example 9

In this experiment, the protective effects of a mixture of stabilizers (200 mM ascorbate and 200 mM glycylglycine) on gamma irradiated liquid anti-IgG1 monoclonal antibody were evaluated.

Methods

Vials were prepared containing 0.335 mg/ml of anti-IgG1 or 0.335 mg/ml of anti-IgG1+200 mM ascorbate+200 mM Gly-Gly. The liquid samples were gamma irradiated to 45 kGy at 4° C. at a rate of 1.752 kGy/hr.

ELISA assays were performed as follows. Two microtitre plates were coated with Human IgG1, Lambda Purified Myeloma Protein at 2 μg/ml, and stored overnight at 4° C. The next day, an ELISA technique was performed using the standard reagents used in the Anti-Insulin ELISA. Following a one hour block, a 10 μg/ml dilution of each sample set was added to the first column of the plate and then serially diluted 3-fold through column 12. Incubation was then performed for one hour at 37° C. Next, a 1:8,000 dilution was made of the secondary antibody, Phosphatase-Labeled Goat Anti-Mouse IgG was added, and incubation was performed for one hour at 37° C. Sigma 104-105 Phosphatase Substrate was added, color was allowed to develop for about 15 minutes, and the reaction was stopped by adding 0.5 M NaOH. Absorbance was measured at 405 nm-620 nm.

Results

Samples irradiated of liquid anti-IgG1 antibody to 45 kGy alone retained essentially no antibody activity. In contrast, samples of liquid anti-IgG1 antibody irradiated to 45 kGy in the presence of the stabilizer mixture (200 mM ascorbate+200 mM Gly-Gly) retained 44% of antibody activity, more than was seen with ascorbate alone.

### Example 10

In this experiment, the protective effects of 20 mM glycylglycine and 20 mM ascorbate on gamma irradiated freeze-dried anti-Ig Lambda Light Chain monoclonal antibody were evaluated.

Methods

Vials containing 20 μg of liquid anti-Ig Lambda Light Chain monoclonal antibody and either 1% bovine serum albumin alone or 1% BSA plus 20 mM ascorbate and 20 mM Gly-Gly were freeze-dried, and irradiated to 45 kGy at a dose rate of 1.741 kGy/hr at 3.8° C.

ELISA assays were performed as follows. Four microtitre plates were coated with Human IgG1, Lambda Purified Myeloma Protein at 2 μg/ml, and stored overnight at 4° C. The next day, an ELISA technique was performed using the standard reagents used in the Anti-Insulin ELISA. Following a one hour block, a 10 μg/ml dilution of each sample set was added to the first column of the plate and then serially diluted 3-fold through column 12. Incubation was then performed for one hour at 37° C. Next, a 1:8,000 dilution was made of the secondary antibody, Phosphatase-Labeled Goat Anti-Mouse IgG was added, and incubation was performed for one hour at 37° C. Sigma 104-105 Phosphatase Substrate was added, color was allowed to develop for about 15 minutes, and the reaction was stopped by adding 0.5 M NaOH. Absorbance was measured at 405 nm-620 nm.

Results

Samples of freeze-dried anti-Ig Lambda Light Chain monoclonal antibody gamma irradiated to 45 kGy with 1% BSA alone retained only 55% of antibody activity. In contrast, samples of freeze-dried anti-Ig Lambda Light Chain monoclonal antibody irradiated to 45 kGy in the presence of the stabilizer mixture (20 mM ascorbate and 20 mM Gly-Gly) retained 76% of antibody activity.

### Example 11

In this experiment, the protective effects of ascorbate and glycylglycine, alone or in combination, on gamma irradiated freeze-dried anti-IgG1 monoclonal antibody were evaluated.

Methods

Vials containing 77.6 μg of anti-IgG1 monoclonal antibody, 1% human serum albumin, and one of 20 mM ascorbate, 20 mM Gly-Gly, or 20 mM ascorbate and 20 mM Gly-Gly, were lyophilized, and gamma irradiated to 47.4 to 51.5 kGy at a dose rate of 1.82 to 1.98 kGy/hr at 4° C.

ELISA assays were performed as follows. Four microtitre plates were coated with Human IgG1, Lambda Purified Myeloma Protein at 2 μg/ml, and stored overnight at 4° C. The next day, an ELISA technique was performed using the standard reagents used in the Anti-Insulin ELISA. Following a one hour block, a 7.75 μg/ml dilution of each sample set was added to the first column of the plate and then serially diluted 3-fold through column 12. Incubation was then performed for one hour at 37° C. Next, a 1:8,000 dilution was made of the secondary antibody, Phosphatase-Labeled Goat Anti-Mouse IgG was added, and incubation was performed for one hour at 37° C. Sigma 104-105 Phosphatase Substrate was added, color was allowed to develop for about 15 minutes, and the reaction was stopped by adding 0.5 M NaOH. Absorbance was measured at 405 nm-620 nm.

Results

Samples of freeze-dried monoclonal anti-IgG1 with 1% human serum albumin retained 62% of antibody activity following gamma irradiation when no stabilizers were present. In contrast, samples of freeze-dried monoclonal anti-IgG1 with 1% human serum albumin and the stabilizer mixture retained 85.3% of antibody activity.

### Example 12

In this experiment, the protective effect of a stabilizer mixture (200 mM ascorbate and 200 mM Gly-Gly) on anti-insulin monoclonal immunoglobulin (50 mg/ml) supplemented with 0.1% human serum albumin (HSA) exposed to gamma irradiation up to 100 kGy was evaluated.

Methods

Samples were irradiated at a dose rate of 0.458 kGy/hr to a total dose of 25, 50 or 100 kGy at ambient temperature (20-25° C).

Monoclonal immunoglobulin activity was determined by a standard ELISA protocol. Maxisorp plates were coated with human recombinant insulin at 2.5 μg/ml overnight at 4° C. The plate was blocked with 380 μl of blocking buffer (PBS, pH 7.4, 2% BSA) for two hours at 37° C. and then washed three times with wash buffer (TBS, pH 7, 0.05% TWEEN 20). Serial 3-fold dilutions were performed. Plates were incubated for one hour at 37° C. with agitation and then washed six times with a wash buffer. Phosphatase-labelled goat anti-mouse IgG (H+L) was diluted to 50 ng/ml in binding buffer and 100 μl was added to each well. The plate was incubated for one hour at 37° C. with agitation and washed eight times with wash buffers. One hundred μl of Sigma-104 substrate (1 mg/ml in DEA buffer) was added to each well and reacted at room temperature. The plate was read on a Multiskan MCC/340 at 405 nm-620 nm.

Results

Samples of anti-insulin monoclonal immunoglobulin supplemented with 1% HSA lost all binding activity when gamma irradiated to 25 kGy. In contrast, samples containing a combination of ascorbate and Gly-Gly retained about 67% of binding activity when irradiated to 25 kGy, 50% when irradiated to 50 kGy and about 33% when irradiated to 100 kGy.

### Example 13

In this experiment, the protective effect of the combination of ascorbate, urate and trolox on gamma irradiated immobilized anti-insulin monoclonal immunoglobulin was evaluated.

Methods

The stabilizer mixture of 200 mM ascorbate (Aldrich 26,855-0, prepared as 2M stock solution in water), 300 FM urate (Sigma U-2875 m, prepared as a 2 mM stock solution in water) and 200 FM trolox (Aldrich 23,681-2, prepared as a 2 mM stock solution in PBS, pH 7.4) was prepared as a solution in PBS pH 7.4 and added to each sample being irradiated. Samples were irradiated to a total dose of 45 kGy at a dose rate of 1.92 kGy/hr at 4° C.

Monoclonal immunoglobulin activity was determined by a standard ELISA protocol. Maxisorp plates were coated with human recombinant insulin at 2 μg/ml overnight at 4° C. The plate was blocked with 200 μl of blocking buffer (PBS, pH 7.4, 2% BSA) for two hours at 37° C. and then washed six times with wash buffer (TBS, pH 7, 0.05% TWEEN 20). Samples were re-suspended in 500 μl of high purity water (100 ng/μl), diluted to 5 μg/ml in a 300 μl U-bottomed plate coated for either overnight or two hours with blocking buffer. Serial 3-fold dilutions were performed, with a final concentration of 0.0022 μg/ml. Plates were incubated for one hour at 37° C. with agitation and then washed six times with a wash buffer. Phosphatase-labelled goat anti-mouse IgG (H+L) was diluted to 50 ng/ml in binding buffer and 100 μl was added to each well. The plate was incubated for one hour at 37° C. with agitation and washed six times with wash buffers. One hundred μl of Sigma-104 substrate (1 mg/ml in DEA buffer) was added to each well and reacted at room temperature. The plate was read on a Multiskan MCC/340 at 405 nM with the 620 nM absorbance subtracted.

Results

Samples of immobilized anti-insulin monoclonal immunoglobulin lost all binding activity when gamma irradiated to 45 kGy. In contrast, samples containing the stabilizer mixture (ascorbate/urate/trolox) retained about 75% of binding activity following gamma irradiation to 45 kGy.

### Example 14

In this experiment, the protective effect of the combination of L-carnosine and ascorbate on gamma irradiated immobilized anti-insulin monoclonal immunoglobulin was evaluated.

Methods

L-carnosine was prepared as a solution in PBS pH 8-8.5 and added to each sample being irradiated across a range of concentration (25 mM, 50 mM, 100 mM or 200 mM). Ascorbate (either 50 mM or 200 mM) was added to some of the samples prior to irradiation. Samples were irradiated at a dose rate of 1.92 kGy/hr to a total dose of 45 kGy at 4° C.

Monoclonal immunoglobulin activity was determined by a standard ELISA protocol. Maxisorp plates were coated with human recombinant insulin at 2 μg/ml overnight at 4° C. The plate was blocked with 200 μl of blocking buffer (PBS, pH 7.4, 2% BSA) for two hours at 37° C. and then washed six times with wash buffer (TBS, pH 7, 0.05% TWEEN 20). Samples were re-suspended in 500 μl of high purity water (100 ng/μl), diluted to 5 μg/ml in a 300 μl U-bottomed plate coated for either overnight or two hours with blocking buffer. Serial 3-fold dilutions were performed, with a final concentration of 0.0022 μg/ml. Plates were incubated for one hour at 37° C. with agitation and then washed six times with a wash buffer. Phosphatase-labelled goat anti-mouse IgG (H+L) was diluted to 50 ng/ml in binding buffer and 100 μl was added to each well. The plate was incubated for one hour at 37° C. with agitation and washed six times with wash buffers. One hundred μl of Sigma-104 substrate (1 mg/ml in DEA buffer) was added to each well and reacted at room temperature. The plate was read on a Multiskan MCC/340 at 405 nM with the 620 nM absorbance subtracted.

Results

Samples of immobilized anti-insulin monoclonal immunoglobulin lost all binding activity when gamma irradiated to 45 kGy. In contrast, samples containing at least 50 mM L-carnosine and 50 mM ascorbate retained about 50% of binding activity following gamma irradiation to 45 kGy.

### Example 15

In this experiment, the protective effects of a number of stabilizer mixtures on gamma irradiated lyophilized Factor VIII were evaluated.

Methods

Samples containing Factor VIII and the stabilizer mixtures of interest (cysteine and ascorbate; N-acetyl-cysteine and ascorbate; or L-carnosine and ascorbate) were lyophilized and stoppered under vacuum. Samples were irradiated at a dose rate of 1.9 kGy/hr to a total dose of 45 kGy at 4° C. Following irradiation, samples were reconstituted with water containing BSA (125 mg/ml) and Factor VIII activity was determined by a one-stage clotting assay using an MLA Electra 1400C Automatic Coagulation Analyzer.

Results

Factor VIII samples containing no stabilizer mixture retained only 32.5% of Factor VIII clotting activity following gamma irradiation to 45 kGy. In contrast, Factor VIII samples containing cysteine and ascorbate retained 43.3% of Factor VIII clotting activity following irradiation. Similarly, Factor VIII samples containing N-acetyl-cysteine and ascorbate or L-carnosine and ascorbate retained 35.5% and 39.8%, respectively, of Factor VIII clotting activity following irradiation to 45 kGy.

### Example 16

In this experiment, the protective effects of 1.5 mM uric acid in the presence of varying amounts of ascorbate on gamma irradiated immobilized anti-insulin monoclonal antibodies were evaluated.

Methods

Maxisorp Immuno microtitre plates were coated with 100 μl of anti-insulin monoclonal antibody (2.5 μg/ml), non-bound antibody was removed by rinsing, 1.5 mM uric acid was added, along with varying amounts (5, 10, 15, 20, 25, 30, 35, 40, 50, 60, 70, 80, 90, 100, 120, 140, 160, 180, 200, 300, 400 and 500 mM) of ascorbate, and were gamma irradiated to 45 kGy at a dose rate of 1.9 kGy/hr at 4° C.

Anti-insulin antibody binding was evaluated by the following procedure. Microtitre plates with anti-insulin monoclonal antibody immobilized therein were incubated and rinsed twice with full volumes of phosphate buffered saline (pH 7.4). Non-specific binding sites were blocked with full volumes of blocking buffer (PBS+2% bovine serum albumin) and 2 hours of incubation at 37° C. The wells were then washed 3 times with TBST (TBS pH 7.4, with 0.05% Tween 20), and to each well was added 100 μl of 10 ng/ml insulin-biotin in binding buffer (0.25% bovine serum albumin in PBS, pH 7.4). The titre plate was then covered/sealed and incubated one hour with shaking at 37° C. The microtitre plates where then washed with TBST for 4 sets of 2 washes/set, with about a 5 minute sitting period allowed between each set. Then, 100 μl of 25 ng/ml phosphatase-labeled Streptavidin was added to each well, the microtitre plate covered/sealed, and incubated at 37° C. with shaking for one hour. The microtitre plates were then washed with TBST for 4 sets of 2 washes per set, with about a 5 minute sitting period allowed between each set. To each well was then added 100 μl of 1 mg/ml Sigma 104 phosphatase substrate in DEA buffer (per liter: 97 ml of diethanolamine, 0.1 g MgCl2.6H2O, 0.02% sodium azide), and the plates incubated at ambient temperature with nutating. Absorbance was then measured at 405 nm-620 nm for each well.

Results

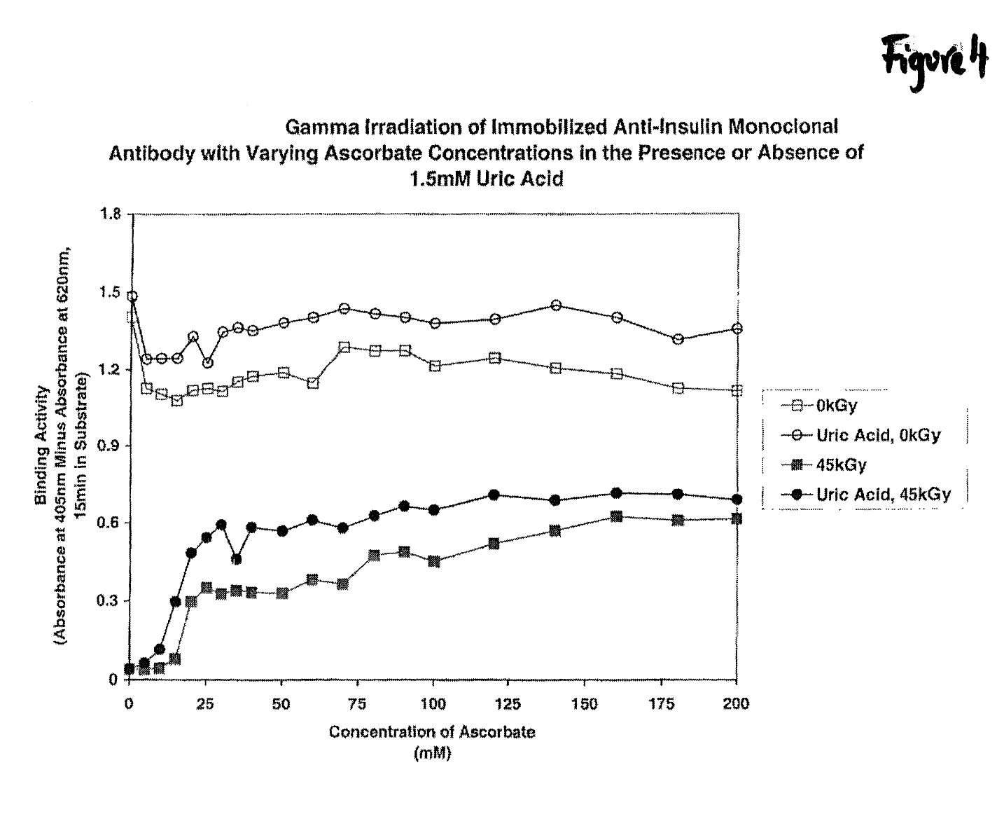

As shown in FIG. 4, the stabilizer mixture of uric acid and ascorbate provided greater protection, as determined by activity retained following irradiation, than ascorbate alone across the range of concentrations employed. Moreover, with ascorbate alone, maximal protection was achieved at a concentration of about 50 mM ascorbate, whereas with the addition of 1.5 mM uric acid, maximal protection was achieved at a concentration of about 30 mM ascorbate.

### Example 17

In this experiment, the protective effects of 2.25 mM uric acid in the presence of varying amounts of ascorbate on gamma irradiated immobilized anti-insulin monoclonal antibodies were evaluated.

Methods

Maxisorp Immuno microtitre plates were coated with 100 μl of anti-insulin monoclonal antibody (2.5 μg/ml), non-bound antibody was removed by rinsing, 1.5 mM uric acid was added, along with varying amounts (5, 10, 15, 20, 25, 30, 35, 40, 50, 60, 70, 80, 90, 100, 120, 140, 160, 180, 200, 300, 400 and 500 mM) of ascorbate, and were gamma irradiated to 45 kGy at a dose rate of 1.9 kGy/hr at 4° C.

Anti-insulin antibody binding was evaluated by the following procedure. Microtitre plates with anti-insulin monoclonal antibody immobilized therein were incubated and rinsed twice with full volumes of phosphate buffered saline (pH 7.4). Non-specific binding sites were blocked with full volumes of blocking buffer (PBS+2% bovine serum albumin) and 2 hours of incubation at 37° C. The wells were then washed 3 times with TBST (TBS pH 7.4, with 0.05% Tween 20), and to each well was added 100 μl of 10 ng/ml insulin-biotin in binding buffer (0.25% bovine serum albumin in PBS, pH 7.4). The titre plate was then covered/sealed and incubated one hour with shaking at 37° C. The microtitre plates where then washed with TBST for 4 sets of 2 washes/set, with about a 5 minute sitting period allowed between each set. Then, 100 μl of 25 ng/ml phosphatase-labeled Streptavidin was added to each well, the microtitre plate covered/sealed, and incubated at 37° C. with shaking for one hour. The microtitre plates were then washed with TBST for 4 sets of 2 washes per set, with about a 5 minute sitting period allowed between each set. To each well was then added 100 μl of 1 mg/ml Sigma 104 phosphatase substrate in DEA buffer (per liter: 97 ml of diethanolamine, 0.1 g MgCl2.6H2O, 0.02% sodium azide), and the plates incubated at ambient temperature with nutating. Absorbance was then measured at 405 nm-620 nm for each well.

Results

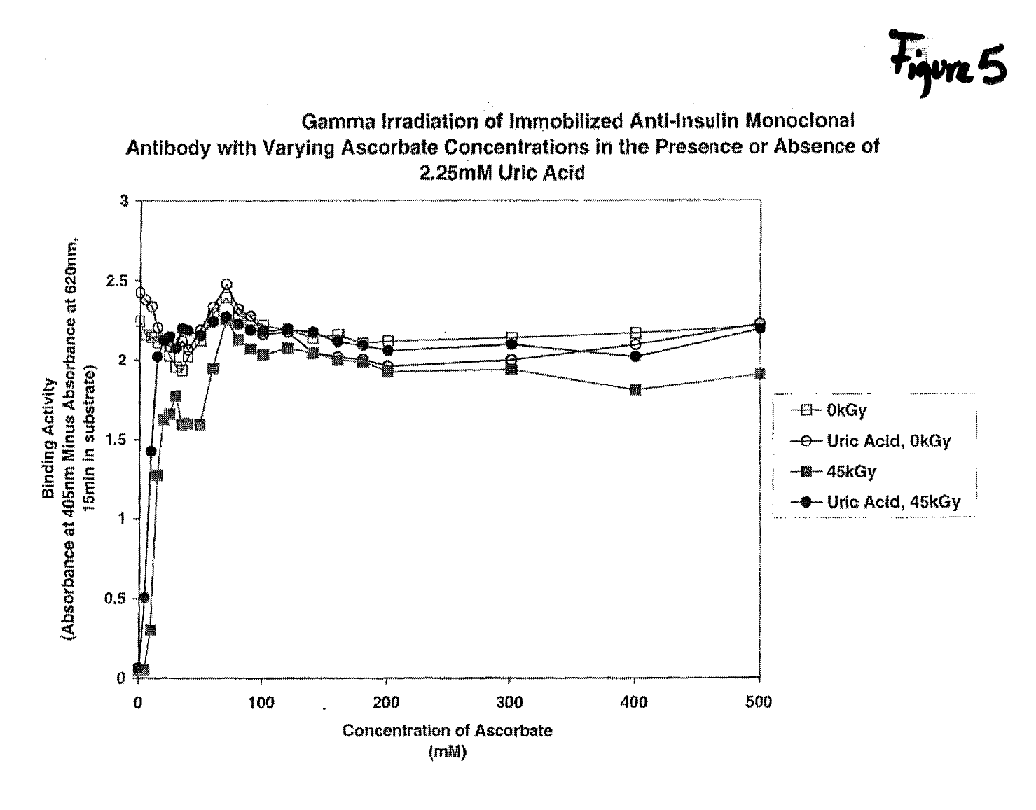

As shown in FIG. 5, the stabilizer mixture of uric acid and ascorbate provided greater protection, as determined by activity retained following irradiation, than ascorbate alone across the range of concentrations employed. Moreover, with ascorbate alone, maximal protection was achieved at a concentration of about 75 mM ascorbate, whereas with the addition of 2.25 mM uric acid, maximal protection (100% activity retained after irradiation) was achieved at a concentration of about 25 mM ascorbate.

### Example 18

In this experiment, the protective effects of various stabilizer mxitures on gamma irradiated lyophilized human coagulation Factor VIII (one step clotting assay) activity.

Methods

Sealed vials containing 12 IU of Baxter Anti-Hemophiliac Factor VIII (Human) and 2.5 mg of bovine serum albumin (total volume 350 μl) were combined with the stabilizer mixture of interest and lyophilized. Lyophilized samples were subjected to gamma irradiation to 45 kGy at a dose rate of 1.9 kGy/hr at 4° C. Following gamma irradiation, each sample was reconstituted in 200 μl of high purity water (from NERL), and assayed for Factor VIII activity using a one-stage clotting assay on an MLA Electra 1400C Automatic Coagulation Analyzer (Hemoliance). The following stabilizer mixtures were tested: 200 mM ascorbate+300 :M uric acid; 300 :M uric acid+200 :M Trolox; and 200 mM ascorbate+300 :M uric acid+200 :M Trolox.

Results

When compared to unirradiated control, irradiated samples containing 200 mM ascorbate+300 :M uric acid exhibited a recovery of 53% of Factor VIII activity. Irradiated samples containing 300 :M uric acid+200 :M Trolox exhibited a recovery of 49% of Factor VIII activity and irradiated samples containing 200 mM ascorbate+300 :M uric acid+200 :M Trolox exhibited a recovery of 53% of Factor VIII activity. In contrast, irradiated samples containing no stabilizer mixture exhibited a recovery of only 38% of Factor VIII activity.

### Example 19

In this experiment, the protective effects of a combination of 200 :M Silymarin+200 mM ascorbate+200 :M Trolox (silymarin cocktail) and a combination of 200 :M Diosmin+200 mM ascorbate+200 :M Trolox (diosmin cocktail), on gamma irradiated lyophilized human anti-hemophiliac clotting Factor VIII (monoclonal) activity were evaluated.

Methods

Aliquots of 200 μl of monoclonal human Factor VIII (21 IU/vial), alone or in combination with the cocktail of interest, were placed in 2 ml vials, frozen at −80EC, and lyophilized. Gamma irradiation to 45 kGy was performed at a dose rate of 1.9 kGy/hr at 4° C. Single-step clotting rates were determined using an MLA Electra 1400C Automatic Coagulation Analyzer (Hemoliance).

Results

Lyophilized Factor VIII irradiated to 45 kGy retained about 18-20% of Factor VIII activity compared to fresh frozen Factor VIII. In contrast, samples containing the diosmin cocktail retained between 40-50% of Factor VIII activity following irradiation to 45 kGy and samples containing the silymarin cocktail retained about 25% of Factor VIII activity following irradiation to 45 kGy.

### Example 20

In this experiment, the protective effects of the combination of ascorbate and trolox and the combination of ascorbate, trolox and urate on urokinase enzymatic activity were evaluated as a function of pH in phosphate buffer solution.

Methods

Samples were prepared in 2 ml vials, each containing 1,000 IU of urokinase (Sigma) and 35 μl of 1M phosphate buffer (pH=4, 5, 5.5, 6.0, 6.47, 7, 7.5, 7.8, 8.5 or 9.0). Stabilizer mixtures (a mixture of 100 Fl of 3 mM trolox and 100 Fl of 2 M sodium ascorbate or a mixture of 100 Fl of 3 mM trolox, 100 Fl of 2 M sodium ascorbate and 100 Fl of 3 mM sodium urate) or trolox alone were added and the samples gamma irradiated to 45 kGy at a dose rate of 1.8 kGy/hr at 4 EC. Residual urokinase activity was determined at room temperature at 5 and 25 minutes after commencement of reaction by addition of urokinase colorimetric substrate #1 (CalBiochem). Optical densities were measured at 405 nm, with subtraction of the optical density at 620 nm.

Results

The irradiated samples containing a stabilizer mixture exhibited much greater retention of urokinase activity compared to samples containing only a single stabilizer across the range of pH tested. More specifically, at pH 4, irradiated samples containing trolox/ascorbate (T/A) retained 65.1% of urokinase activity and samples containing trolox/ascorbate/urate (T/A/U) retained 66.2% of urokinase activity. In contrast, at pH 4, samples containing only trolox retained only 5.3% of urokinase activity. The following results were also obtained:

| pH | stabilizer | urokinase activity |
|---|---|---|
| 5.0 | trolox | 13% |
| | T/A | 72.2% |
| | T/A/U | 62.2% |
| 5.5 | trolox | 13% |
| | T/A | 66.7% |
| | T/A/U | 66.3% |
| 6.0 | trolox | 30% |
| | T/A | 61.8% |
| | T/A/U | 61.8% |
| 6.47 | trolox | 30% |
| | T/A | 70.5% |
| | T/A/U | 70.2% |
| 7.0 | trolox | 20% |
| | T/A | 69.5% |
| | T/A/U | 65.9% |
| 7.5 | trolox | 24% |
| | T/A | 72.1% |
| | T/A/U | 64.0% |
| 7.8 | trolox | 28% |
| | T/A | 63.5% |
| | T/A/U | 70.7% |
| 8.5 | trolox | 23% |
| | T/A | 64.4% |
| | T/A/U | 70.2% |
| 9.0 | trolox | 38% |
| | T/A | 71.3% |
| | T/A/U | 68.73% |

### Example 21

In this experiment, the protective effects of the combination of ascorbate and urate on urokinase enzymatic activity were evaluated as a function of pH in phosphate buffer solution.

Methods

Samples were prepared in 2 ml vials, each containing 1,000 IU of urokinase (Sigma) and 35 μl of 1M phosphate buffer (pH=4, 5, 6.0, 6.47, 7, 7.8 or 9.0). A stabilizer mixture of 100 Fl of 2 M sodium ascorbate and 100 Fl of 3 mM sodium urate was added and the samples gamma irradiated to 45 kGy at a dose rate of 1.8 kGy/hr at 4EC. Residual urokinase activity was determined at room temperature at 5 and 25 minutes after commencement of reaction by addition of urokinase colorimetric substrate #1 (CalBiochem). Optical densities were measured at 405 nm, with subtraction of the optical density at 620 nm.

Results

The irradiated samples containing a stabilizer mixture exhibited much greater retention of urokinase activity compared to samples containing only urate across the range of pH tested. More specifically, irradiated samples containing ascorbate/urate retained between 48.97% (at pH 9.0) and 64.01% (at pH 6.47) of urokinase activity, whereas irradiated samples containing only urate retained essentially no urokinase activity.

### Example 22

In this experiment, the protective effects of the combination of ascorbate (200 mM) and Gly-Gly (200 mM) on lyophilized galactosidase preparations were investigated.

Methods

Samples were prepared in glass vials, each containing 300 Fg of a lyophilized glycosidase and either no stabilizer or the stabilizer mixture. Samples were irradiated with gamma radiation to varying total doses (10 kGy, 30 kGy and 50 kGy total dose, at a rate of 0.6 kGy/hr. and a temperature of −60° C.) and then assayed for structural integrity using SDS-PAGE.

Samples were reconstituted with water to a concentration of 1 mg/ml, diluted 1:1 with 2×sample buffer (15.0 ml 4×Upper Tris-SDS buffer (pH 6.8); 1.2 g sodium dodecyl sulfate; 6 ml glycerol; sufficient water to make up 30 ml; either with or without 0.46 g dithiothreitol), and then heated at 80EC for 10 minutes. 10 Fl of each sample (containing 5 Fg of enzyme) were loaded into each lane of a 10% polyacrylamide gel and run on an electrophoresis unit at 125V for about 1.5 hours.

Results

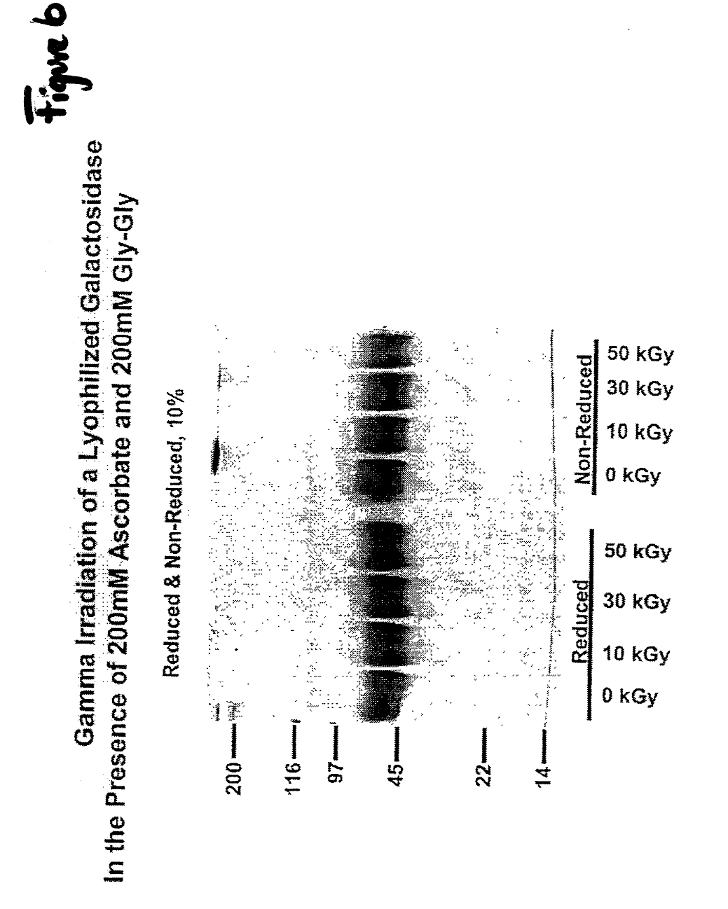

About 80% of the enzyme was recovered following irradiation of the samples containing no stabilizer, with some degradation as shown in FIGS. 6A-6C. Significantly less degradation was observed in the samples containing a combination of ascorbate and glycylglycine as the stabilizer mixture.

### Example 23

In this experiment, the protective effects of ascorbate and lipoic acid on gamma irradiated liquid Thrombin activity were evaluated.

Methods

Two microtitre dilution plates were prepared—one for samples to receive gamma irradiation, and one for control samples (no gamma irradiation)—containing a range of concentrations of ascorbate and lipoic acid. Samples receiving gamma irradiation were irradiated to 45 kGy at a dose rate of 1.788 kGy/hr at 4.2° C.

Thrombin activity was measured by conventional procedure, which was commenced by adding 50 μl of 1600 :M substrate to each 50 μl of sample in a well of a Nunc 96 low protein binding plate, and absorbance was read for 60 minutes at 10 minute intervals.

Results

When both ascorbate and lipoic acid were present, synergistic protective effects were apparent, as is shown by the following data:

| [ascorbate] | [lipoic acid] | | % recovery of Thrombin activity |
|---|---|---|---|
| 0 mM | 100 | mM | 10% |
| 10 mM | 0 | mM | 2% |
| 10 mM | 200-225 | mM | 80.3% |
| 50 mM | 100-175 | mM | 82-85% |
| 100 mM | 10-25 | mM | 78% |
| 100 mM | 0 | mM | 52% |

### Example 24

In this experiment, the protective effects of a combination of ascorbate and lipoic acid on gamma irradiated freeze-dried Thrombin activity were evaluated.

Methods

Two microtitre dilution plates were prepared—one for samples to receive gamma irradiation, and one for control samples (no gamma irradiation)—containing a range of concentrations of ascorbate and lipoic acid. Samples receiving gamma irradiation were irradiated to 45 kGy at a dose rate of 1.78 kGy/hr at 4.80° C.

Thrombin activity was measured by conventional procedure, which was commenced by adding 50 μl of 1600 :M substrate to each 50 μl of sample in a well of a Nunc 96 low protein binding plate, and absorbance was read for 60 minutes at 10 minute intervals.

Results

When both ascorbate and lipoic acid were present, synergistic protective effects were apparent, as is shown by the following data:

| [ascorbate] | [lipoic acid] | % recovery of Thrombin activity |
|---|---|---|
| 0 mM | 0 mM | 54.8% |
| 0 mM | 100 mM | 73.5% |
| 25 mM | 0 mM | 74.5% |
| 2.5 mM | 40 mM | 83.5% |
| 5 mM | 5 mM | 80.3% |
| 5 mM | 10 mM | 84.3% |
| 5 mM | 100 mM | 89.5% |
| 10 mM | 40 mM | 85.% |
| 25 mM | 10 mM | 86.2% |
| 25 mM | 100 mM | 84.7% |

### Example 25

In this experiment, the protective effects of a combination of ascorbate and hydroquinonesulfonic acid (HQ) on gamma irradiated liquid Thrombin were evaluated.

Methods

Two microtitre dilution plates were prepared—one for samples to receive gamma irradiation, and one for control samples (no gamma irradiation)—containing a range of concentrations of ascorbate and hydroquinonesulfonic acid (HQ). Samples receiving gamma irradiation were irradiated to 45 kGy at a dose rate of 1.78 kGy/hr at 3.5-4.9° C.

Thrombin activity was measured by conventional procedure, which was commenced by adding 50 μl of 1600 :M substrate to each 50 μl of sample in a well of a Nunc 96 low protein binding plate, and absorbance was read for 60 minutes at 10 minute intervals.

Results

When both ascorbate and hydroquinonesulfonic acid were present, synergistic protective effects were apparent, as is shown by the following data:

| [ascorbate] | [HQ] | | % recovery of Thrombin activity |
|---|---|---|---|
| 0 mM | 0 | mM | 0% |
| 0 mM | 187.5 | mM | 2% |
| 200 mM | 0 | mM | 59% |
| 200 mM | 187.5 | mM | 68% |
| 50 mM | 187.5 | mM | 70% |
| 50 mM | 100 | mM | 70% |
| 50 mM | 50 | mM | 66.9% |
| 100 mM | 75 | mM | 73% |
| 100 mM | 100 | mM | 73% |
| 200 mM | 25-50 | mM | 72% |

### Example 26

In this experiment, the protective effects of a combination of ascorbate (200 FM), urate (0.3 mM) and trolox (0.2 mM) on gamma irradiated liquid Thrombin were evaluated.

Methods

Samples were prepared of thrombin (5000 U/ml) and either no stabilizer or the stabilizer mixture of interest. Samples receiving gamma irradiation were irradiated to 45 kGy at a dose rate of 1.852 kGy/hr at 4° C.

Following irradiation, thrombin activity was measured by conventional procedure.

Results

Samples of liquid thrombin containing no stabilizer retained no activity following irradation to 45 kGy. In contrast, samples of liquid thrombin containing the ascorbate/trolox/urate mixture retained about 50% of thrombin activity following irradiation to 45 kGy.

### Example 27

In this experiment, the protective effects of a combination of ascorbate (200 FM), urate (0.3 mM) and trolox (0.2 mM) on gamma irradiated liquid Thrombin were evaluated.

Methods

Samples were prepared of thrombin (5000 U/ml) and either no stabilizer or the stabilizer mixture of interest and, optionally, 0.2% bovine serum albumin (BSA). Samples receiving gamma irradiation were irradiated to 45 kGy at a dose rate of 1.852 kGy/hr at 4° C.

Following irradiation, thrombin activity was measured by conventional procedure.

Results

Samples of liquid thrombin containing no stabilizer or BSA alone retained no activity following irradation to 45 kGy. In contrast, samples of liquid thrombin containing the ascorbate/trolox/urate mixture retained about 50% of thrombin activity following irradiation to 45 kGy. Moreover, samples of liquid thrombin containing ascorbate/trolox/urate and BSA retained between 55 and 78.5% of thrombin activity following irradiation to 45 kGy.

Having now fully described this invention, it will be understood to those of ordinary skill in the art that the methods of the present invention can be carried out with a wide and equivalent range of conditions, formulations, and other parameters without departing from the scope of the invention or any embodiments thereof.

All patents and publications cited herein are hereby fully incorporated by reference in their entirety. The citation of any publication is for its disclosure prior to the filing date and should not be construed as an admission that such publication is prior art or that the present invention is not entitled to antedate such publication by virtue of prior invention.

The foregoing embodiments and advantages are merely exemplary and are not to be construed as limiting the present invention. The present teaching can be readily applied to other types of apparatuses. The description of the present invention is intended to be illustrative, and not to limit the scope of the claims. Many alternatives, modifications, and variations will be apparent to those skilled in the art.

The foregoing embodiments and advantages are merely exemplary and are not to be construed as limiting the present invention. The present teachings can be readily applied to other types of apparatuses. The description of the present invention is intended to be illustrative, and not to limit the scope of the claims. Many alternatives, modifications, and variations will be apparent to those skilled in the art. In the claims, means-plus-function clauses are intended to cover the structures described herein as performing the recited function and not only structural equivalents but also equivalent structures.

What is claimed is:

1. A method for sterilizing a biological material that is sensitive to radiation, said method comprising:
(i) adding to said biological material at least one stabilizer mixture in an amount effective to protect said biological material from said radiation; and
(ii) irradiating said biological material with a suitable radiation at an effective rate for a time effective to sterilize said biological material.

2. A method for sterilizing a biological material that is sensitive to radiation, said method comprising:
(i) reducing the residual solvent content of said biological material;
(ii) adding to said biological material at least one stabilizer mixture; and
(iii) irradiating said biological material with a suitable radiation at an effective rate for a time effective to sterilize said biological material, wherein the level of said residual solvent content and the amount of said stabilizer mixture are together effective to protect said biological material from said radiation, and further wherein steps (i) and (ii) may be performed in inverse order.

3. A method for sterilizing a biological material that is sensitive to radiation, said method comprising:
(i) reducing the temperature of said biological material;
(ii) adding to said biological material at least one stabilizer mixture; and
(iii) irradiating said biological material with a suitable radiation at an effective rate for a time effective to sterilize said biological material, wherein the temperature and the amount of said stabilizer mixture are together effective to protect said biological material from said radiation, and further wherein steps (i) and (ii) may be performed in inverse order.

4. The method according to claim 2, wherein said solvent is water.

5. The method according to claim 4, wherein said residual water content is reduced by the addition of an organic solvent.

6. The method according to claim 2, wherein said solvent is an organic solvent.

7. The method according to claim 2, wherein said biological material is suspended in an organic solvent following reduction of said residual solvent content.

8. The method according to claim 1, 2, 3 or 86, wherein said effective rate is not more than about 3.0 kGy/hour.

9. The method according to claim 1, 2, 3 or 86, wherein said effective rate is not more than about 2.0 kGy/hr.

10. The method according to claim 1, 2, 3 or 86, wherein said effective rate is not more than about 1.0 kGy/hr.

11. The method according to claim 1, 2, 3 or 86, wherein said effective rate is not more than about 0.3 kGy/hr.

12. The method according to claim 1, 2, 3 or 86, wherein said effective rate is more than about 3.0 kGy/hour.

13. The method according to claim 1, 2, 3 or 86, wherein said effective rate is at least about 6.0 kGy/hour.

14. The method according to claim 1, 2, 3 or 86, wherein said effective rate is at least about 18.0 kGy/hour.

15. The method according to claim 1, 2, 3 or 86, wherein said effective rate is at least about 30.0 kGy/hour.

16. The method according to claim 1, 2, 3 or 86, wherein said effective rate is at least about 45 kGy/hour.

17. The method according to claim 1, 2, 3 or 86, wherein said biological material is maintained in a low oxygen atmosphere.

18. The method according to claim 1, 2, 3 or 86, wherein said biological material is maintained in an atmosphere comprising at least one noble gas.

19. The method according to claim 18, wherein said noble gas is argon.

20. The method according to claim 1, 2, 3 or 86, wherein said biological material is maintained in a vacuum.

21. The method according to claim 2, wherein said residual solvent content is reduced by a method selected from the group consisting of lyophilization, drying, concentration, addition of solute, evaporation, chemical extraction, spray-drying, and vitrification.

22. The method according to claim 2, wherein said residual solvent content is less than about 15%.

23. The method according to claim 2, wherein said residual solvent content is less than about 3%.

24. The method according to claim 2, wherein said residual solvent content is less than about 2%.

25. The method according to claim 2, wherein said residual solvent content is less than about 1%.

26. The method according to claim 2, wherein said residual solvent content is less than about 0.5%.

27. The method according to claim 2, wherein said residual solvent content is less than about 0.08%.

28. The method according to claim 1, 2, 3 or 86, wherein at least one sensitizer is added to said biological material prior to said step of irradiating said biological material.

29. The method according to claim 1, 2, 3 or 86, wherein said stabilizer mixture comprises at least three stabilizers.

30. The method according to claim 1, 2, 3 or 86, wherein said stabilizer mixture contains at least one antioxidant.

31. The method according to claim 1, 2, 3 or 86, wherein said stabilizer mixture contains at least one free radical scavenger.

32. The method according to claim 1, 2, 3 or 86, wherein said stabilizer mixture contains at least one combination stabilizer.

33. The method according to claim 1, 2, 3 or 86, wherein said stabilizer mixture contains at least one ligand.

34. The method according to claim 33, wherein said ligand is heparin.

35. The method according to claim 1, 2, 3 or 86, wherein said stabilizer mixture contains at least one stabilizer that reduces damage due to reactive oxygen species.

36. The method according to claim 1, 2, 3 or 86, wherein said stabilizer mixture contains at least one stabilizer selected from the group consisting of: ascorbic acid or a salt or ester thereof; glutathione; vitamin E or a derivative thereof; albumin; sucrose; glycylglycine; L-carnosine; cysteine; silimarin; diosmin; hydroquinonesulfonic acid; 6-hydroxy-2,5,7,8-tetramethylchroman-2-carboxylic acid; uric acid or a salt or ester thereof; methionine; histidine; N-acetyl cysteine; lipoic acid; sodium formaldehyde sulfoxylate; gallic acid or a derivative thereof; propyl gallate and mixtures of two or more thereof.

37. The method according to claim 36, wherein said mixtures of two or more additional stabilizers are selected from the group consisting of: mixtures of ascorbic acid, or a salt or ester thereof, and uric acid, or a salt or ester thereof; mixtures of ascorbic acid, or a salt or ester thereof, and 6-hydroxy-2,5,7,8-tetramethylchroman-2-carboxylic acid; mixtures of ascorbic acid, or a salt or ester thereof, uric acid, or a salt or ester thereof, and 6-hydroxy-2,5,7,8-tetramethylchroman-2-carboxylic acid; mixtures of ascorbic acid, or a salt or ester thereof, uric acid, or a salt or ester thereof, and 6-hydroxy-2,5,7,8-tetramethylchroman-2-carboxylic acid, and albumin; mixtures of ascorbic acid, or a salt or ester thereof, uric acid, or a salt or ester thereof, and 6-hydroxy-2,5,7,8-tetramethylchroman-2-carboxylic acid, albumin and sucrose; mixtures of ascorbic acid, or a salt or ester thereof, and glycylglycine; mixtures of ascorbic acid, or a salt or ester thereof, glycylglycine and albumin; mixtures of ascorbic acid, or a salt or ester thereof and L-carnosine; mixtures of ascorbic acid, or a salt or ester thereof and cysteine; mixtures of ascorbic acid, or a salt or ester thereof and N-acetyl cysteine; mixtures of ascorbic acid, or a salt or ester thereof, uric acid, or a salt or ester thereof, and 6-hydroxy-2,5,7,8-tetramethylchroman-2-carboxylic acid, and silymarin; mixtures of ascorbic acid, or a salt or ester thereof, uric acid, or a salt or ester thereof, and 6-hydroxy-2,5,7,8-tetramethylchroman-2-carboxylic acid, and diosmin; mixtures of ascorbic acid, or a salt or ester thereof, uric acid, or a salt or ester thereof, and lipoic acid; mixtures of ascorbic acid, or a salt or ester thereof, uric acid, or a salt or ester thereof, and hydroquinonesulfonic acid and mixtures of uric acid, or a salt or ester thereof; lipoic acid; sodium formaldehyde sulfoxylate; gallic acid or a derivative thereof; propyl gallate and 6-hydroxy-2,5,7,8-tetramethylchroman-2-carboxylic acid.

38. The method according to claim 1, 2, 3 or 86, wherein said stabilizer mixture comprises ascorbic acid or a salt or ester thereof.

39. The method according to claim 1, 2, 3 or 86, wherein said radiation is corpuscular radiation or electromagnetic radiation, or a mixture thereof.

40. The method according to claim 39, wherein said electromagnetic radiation is selected from the group consisting of radio waves, microwaves, visible and invisible light, ultraviolet light, x-ray radiation, gamma radiation and combinations thereof.

41. The method according to claim 1, 2, 3 or 86, wherein said radiation is gamma radiation.

42. The method according to claim 1, 2, 3 or 86, wherein said radiation is E-beam radiation.

43. The method according to claim 1, 2, 3 or 86, wherein said radiation is visible light.

44. The method according to claim 1, 2, 3 or 86, wherein said radiation is ultraviolet light.

45. The method according to claim 1, 2, 3 or 86, wherein said radiation is x-ray radiation.

46. The method according to claim 1, 2, 3 or 86, wherein said radiation is polychromatic visible light.

47. The method according to claim 1, 2, 3 or 86, wherein said radiation is infrared.

48. The method according to claim 1, 2, 3 or 86, wherein said radiation is a combination of one or more wavelengths of visible and ultraviolet light.

49. The method according to claim 1, 2, 3 or 86, wherein said irradiation is conducted at ambient temperature.

50. The method according to claim 1, 2, 3 or 86, wherein said irradiation is conducted at a temperature below ambient temperature.

51. The method according to claim 1, 2, 3 or 86, wherein said irradiation is conducted below the freezing point of said biological material.

52. The method according to claim 1, 2, 3 or 86, wherein said irradiation is conducted below the eutectic point of said biological material.

53. The method according to claim 1, 2, 3 or 86, wherein said irradiation is conducted at a temperature above ambient temperature.

54. A composition comprising at least one biological material and at least one stabilizer mixture in an amount effective to preserve said biological material for its intended use following sterilization with radiation.

55. The composition according to claim 54, wherein said stabilizer mixture contains at least one stabilizer selected from the group consisting of: ascorbic acid or a salt or ester thereof; glutathione; 6-hydroxy-2,5,7,8-tetramethylchroman-2-carboxylic acid; uric acid or a salt or ester thereof; methionine; histidine; N-acetyl cysteine; diosmin; silymarin; lipoic acid; sodium formaldehyde sulfoxylate; gallic acid or a derivative thereof, propyl gallate, vitamin E or a derivative thereof; albumin; sucrose; glycylglycine; L-carnosine; cysteine; hydroquinonesulfonic acid; a mixture of ascorbic acid, or a salt or ester thereof, and uric acid, or a salt or ester thereof; a mixture of ascorbic acid, or a salt or ester thereof, and 6-hydroxy-2,5,7,8-tetramethylchroman-2-carboxylic acid; a mixture of ascorbic acid, or a salt or ester thereof, uric acid, or a salt or ester thereof, and 6-hydroxy-2,5,7,8-tetramethylchroman-2-carboxylic acid; and a mixture of uric acid, or a salt or ester thereof and 6-hydroxy-2,5,7,8-tetramethylchroman-2-carboxylic acid, mixtures of ascorbic acid, or a salt or ester thereof, uric acid, or a salt or ester thereof, and 6-hydroxy-2,5,7,8-tetramethylchroman-2-carboxylic acid, and albumin; mixtures of ascorbic acid, or a salt or ester thereof, uric acid, or a salt or ester thereof, and 6-hydroxy-2,5,7,8-tetramethylchroman-2-carboxylic acid, albumin and sucrose; mixtures of ascorbic acid, or a salt or ester thereof, and glycylglycine; mixtures of ascorbic acid, or a salt or ester thereof, glycylglycine and albumin; mixtures of ascorbic acid, or a salt or ester thereof and L-carnosine; mixtures of ascorbic acid, or a salt or ester thereof and cysteine; mixtures of ascorbic acid, or a salt or ester thereof and N-acetyl cysteine; mixtures of ascorbic acid, or a salt or ester thereof, uric acid, or a salt or ester thereof, and 6-hydroxy-2,5,7,8-tetramethylchroman-2-carboxylic acid, and silymarin; mixtures of ascorbic acid, or a salt or ester thereof, uric acid, or a salt or ester thereof, and 6-hydroxy-2,5,7,8-tetramethylchroman-2-carboxylic acid, and diosmin; mixtures of ascorbic acid, or a salt or ester thereof, uric acid, or a salt or ester thereof, and lipoic acid; mixtures of ascorbic acid, or a salt or ester thereof, uric acid, or a salt or ester thereof, and hydroquinonesulfonic acid.

56. The composition of claim 54, wherein the residual solvent content of said biological material is sufficiently low to preserve said biological material, during sterilization by irradiation, for its intended use following sterilization with radiation.

57. The composition of claim 56, wherein said residual solvent content is less than about 15%.

58. The composition of claim 56, wherein said residual solvent content is less than about 10%.

59. The composition of claim 56, wherein said residual solvent content is less than about 5%.

60. The composition of claim 56, wherein said residual solvent content is less than about 2%.

61. The composition of claim 56, wherein said residual solvent content is less than about 1%.

62. The composition of claim 56, wherein said residual solvent content is less than about 0.5%.

63. The composition of claim 56, wherein said residual solvent content is less than about 0.08%.

64. The composition of claim 56, wherein said biological material is glassy or vitrified.

65. The composition of claim 54, wherein said biological material is selected from the group consisting of monoclonal immunoglobulins, polyclonal immunoglobulins, glycosidases, sulfatases, urokinase and Factor VIII.

66. The composition of claim 56, wherein the concentration of said biological material is at least about 0.5%.

67. The composition of claim 56, wherein the concentration of said biological material is at least about 1%.

68. The composition of claim 56, wherein the concentration of said biological material is at least about 5%.

69. The composition of claim 56, wherein the concentration of said biological material is at least about 10%.

70. The composition of claim 56, wherein the concentration of said biological material is at least about 15%.

71. The composition of claim 56, wherein the concentration of said biological material is at least about 20%.

72. The composition of claim 56, wherein the concentration of said biological material is at least about 25%.

73. The composition of claim 56, wherein the concentration of said biological material is at least about 50%.

74. A method of treating a disease or deficiency in a mammal comprising administering to a mammal in need thereof an effective amount of a biological preparation which has been sterilized according to the method according to claim 1, 2, 3, or 86.

75. The method according to claim 74, wherein said mammal is a human.

76. The method according to claim 74, wherein said deficiency is Factor VIII deficiency.

77. The method according to claim 74, wherein said disease responds to the administration of urokinase.

78. The method according to claim 74, wherein said disease responds to the administration of thrombin.

79. The method according to claim 74, wherein said deficiency is a glucosidase deficiency.

80. The method according to claim 74, wherein said deficiency is a galactosidase deficiency.

81. The method according to claim 80, wherein said deficiency is a Fabry's Disease.

82. The method according to claim 74, wherein said deficiency is a sulfatase deficiency.

83. The method according to claim 74, wherein said deficiency is an Immunoglobulin deficiency.

84. The method according to claim 74, wherein said disease responds to the administration of an Immunoglobulin.

85. The method according to claim 74, wherein said disease responds to the administration of Factor VIII.

86. A method for sterilizing a biological material that is sensitive to radiation, said method comprising:
(i) reducing the residual solvent content of said biological material;
(ii) adding to said biological material at least one stabilizer mixture
(iii) reducing the temperature of said biological material; and
(iv) irradiating said biological material with a suitable radiation at an effective rate for a time effective to sterilize said biological material, wherein the temperature and the amount of said stabilizer mixture are together effective to protect said biological material from said radiation, and further wherein steps (i), (ii) and (iii) may be performed in any order.

87. The method according to claim 2, wherein said residual solvent content is less than about 10%.
